(19) United States
(12) Patent Application Publication
(10) Pub. No.: US 2008/0194912 A1
(43) Pub. Date: Aug. 14, 2008

(54) Electronically Controlled Ingestible Capsule for Sampling Fluids in Alimentary Tract

(75) Inventors: Karen I. Trovato, Putnam Valley, NY (US); Judy R. Naamat, Far Rockaway, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/814,176

(22) PCT Filed: Jan. 16, 2006
(86) PCT No.: PCT/IB06/50160

§ 371 Date: Jul. 18, 2007

Related U.S. Application Data
(60) Provisional application No. 60/644,538, filed on Jan. 18, 2005.

(51) Int. Cl. A61B 1/00

(52) U.S. Cl. 600/118

(57) ABSTRACT

An ingestible electrical capsule system (1400) for acquiring samples along the alimentary tract of a patient is provided. The capsule system (1400) includes a housing (102) having at least one aperture (970); at least one impervious collection chamber (1402) disposed within the housing (102) and having at least one aperture in fluid communication with a respective aperture of the housing (102); and at least one closure member (966). Individual closure members (966) are associated with a respective aperture of the at least one collection chamber (1402), wherein the individual closure members (966) are actuatable between an open state for permitting flow of the fluid through the respective closure member into the associated collection chamber (1402) for acquiring a sample of ambient fluid, and a closed state for substantially blocking flow of fluid into and out of the associated collection chamber (1402) for storing the acquired sample. The capsule system (1400) further includes control circuitry (906) for controlling actuation of the at least one closure member (966).

The present invention relates generally to an electronically controlled capsule. More particularly, it relates to a system and method for an ingestible electronically controlled capsule for sampling fluids of the alimentary tract of a patient.

A medicament is generally administered as a capsule or a liquid to be taken at least one time per day. A person may be required to take or be administered several medicaments each day during the same or different times. This requires that the person or his caregiver maintain a log or remember which medicaments to take or administer at different times during the day.

A medicament, such as aspirin, taken by the person generally traverses the alimentary tract where it is absorbed for treating an ailment or condition. Objects typically pass through the alimentary tract in 20-40 hours. Several medicaments are available as time-release capsules for releasing portions of the medicament into the body at different times. Time-release capsules utilize chemical reactions between chemical substances in the gastrointestinal tract and the coating of the capsules for dissolving and releasing the medicament. Food, particularly proteins and fats, and the gastrointestinal (GI) chemistry affect the speed of the journey of medicaments through the stomach. As such, medicaments, including medicaments available as time-release capsules, do not follow an exact dispensing or dissolving pattern while traveling through the alimentary tract.

For example, one person may have more than a “normal” amount of chemical substances in the gastrointestinal tract due to a condition, an earlier-administered medicament, etc. and therefore, cause the coating of the time-release capsule to react quicker than normal. Accordingly, the medicament is released by the time-release capsule at a faster rate than an intended rate. However, another person may have less than the “normal” amount of chemical substance in the gastrointestinal tract and cause the coating of the time-release capsule to react slower than normal, thereby releasing the medicament at a slower rate than the intended rate.

Further, as with traditional medicaments available in non-time-release form, time-release capsules require a person or caregiver maintain a log or remember which medicaments to take or administer at different times during the day. For example, some medicaments must be taken at bedtime, such as NSAIDS for rheumatoid arthritis, to produce fewer gastrointestinal complications, such as indigestion. Other medicaments, such as the anti-inflammatory corticosteroid medication predisone, can cause insomnia when taken in high doses, and are typically taken in the morning. Still, other medicaments, such as antihistamines, are typically taken in the evening to prepare for symptoms that often occur in the morning.

Furthermore, release of the medicament from the capsule is not controllable for intermittent dispensing of the medicament based on a control factor, such as time or a sensed property and independent of the amount of medication held in the capsule's reservoir. In addition, once ingested, movement of the capsule independent of the movements of the alimentary tract is not controllable.

Current diagnostics methods for sampling internal tissue are invasive and/or limited in the ability to access all areas of the alimentary tract. Furthermore, current diagnostic methods do not provide the ability to automatically and controllably obtain samples at discrete times based on a control factor, such as time or a sensed property. Additionally, current diagnostic methods do not provide a topographical mapping of anatomy within the alimentary tract based on sensory input.

Implantable devices or seeds are known for release of medicament or radiation at the implantation site. However, current implantable devices or seeds do not provide for controllable intermittent dispensing of a medicament or radiation based on a control factor, such as time or a sensed property and independent of the amount of medication or radiation stored by the implant.

The present disclosure provides an electronically controlled capsule or medicament delivery system for delivering or dispensing a medicament according to a preset dispensing timing pattern while traversing through the gastrointestinal tract. The preset dispensing timing pattern is fixed and is not susceptible to a person's physiological processes and conditions, mood, earlier-administered medicaments, etc. The electronically controlled capsule includes control and timing circuitry for controlling the opening and closing of a valve or hatch according to the preset dispensing timing pattern for dispensing a medicament stored within a medicament reservoir of the capsule. The electronically controlled capsule allows a person to take all capsules substantially simultaneously, at say 7:00 am, so that no more capsules are required for the day. Medication that does not fit into one electronically controlled capsule can be coordinated with other electronically controlled capsules for the full day's payload regimen.

According to the present disclosure, all of the medicaments required to be taken during a particular time period, for example, during a 24-hour period, can be provided within one or more electronically controlled capsules which can all be taken at the same time. The electronically controlled capsules can have different dispensing timing patterns, so that a full day's coverage can be obtained. As such, the present disclosure also provides a treatment system for administering two or more medicaments at the same time via the one or more electronically controlled capsules. Each capsule has an independent, preset dispensing timing pattern in order to dispense its medicaments within the body according to a dispensing pattern. The dispensing pattern can be varied from person to person depending on each person's physical condition, age, gender, ailments, etc. Further, at a preset moment in time during the dispensing timing patterns, the electronically controlled capsules present in the body may be programmed to stop dispensing medicament, in the expectation that a new set of capsules will be taken. This prevents accidental overdose by having only the most recently taken capsules dispensing medicament in the body.

The treatment system of the present disclosure enables an individual to take all of his medicaments at substantially the same time, e.g., in the morning or in the evening, and not at different times during a particular time period (e.g., a 24-hour period). The treatment system of the present disclosure further enables a caregiver to administer once per day (i.e., once per a 24-hour period) all of the medicaments for each patient of a hospital or resident of a nursing home (or animals in a shelter or veterinary facility). The system of the present disclosure therefore avoids the need for a caregiver to wake up or otherwise disturb a patient or resident for the sole purpose of administering a medicament, or to track down a patient or resident who may be in a different part of the hospital or nursing home for the sole purpose of administering a medicament. The system of the present disclosure also reduces the overload required for inventorying, ordering, tracking and logging the medicaments.

In another embodiment of the disclosure, an ingestible electrical capsule system for acquiring samples along the alimentary tract of a patient is provided. The capsule system includes a housing having at least one aperture; at least one impervious collection chamber disposed within the housing and having at least one aperture in fluid communication with a respective aperture of the housing; and at least one closure member. Individual closure members are associated with a respective aperture of the at least one collection chamber, wherein the individual closure members are actuatable between an open state for permitting flow of the fluid through the respective closure member into the associated collection chamber for acquiring a sample of ambient fluid, and a closed state for substantially blocking flow of fluid into and out of the associated collection chamber for storing the acquired sample. The capsule system further includes control circuitry for controlling actuation of the at least one closure member.

In a further embodiment of the disclosure, an ingestible electrical capsule system for traversing the alimentary tract of a patient is provided. The capsule system includes housing; at least one expulsion sensor for sensing expulsion or imminent expulsion of the capsule system from the alimentary tract and generating corresponding signals; an alert device for selectively generating an alert; and control circuitry. The control circuitry processes the corresponding signals generated by the expulsion sensor for determining when the corresponding signals are indicative of sensed expulsion or imminent expulsion, and controlling the alert device to generate the alert when it is determined that the corresponding signals indicate sensed expulsion or imminent expulsion.

In still another embodiment of the invention a method is provided for generating an alert upon expulsion or imminent expulsion from the alimentary tract of a device traversing the digestive tract. The method includes the steps of sensing expulsion or imminent expulsion of the capsule system from the alimentary tract and generating corresponding signals; processing the corresponding signals generated by the expulsion sensor;

determining when the corresponding signals are indicative of sensed expulsion or imminent expulsion, and generating an alert when it is determined that the corresponding signals indicate sensed expulsion or imminent expulsion.

Various embodiments of the present disclosure will be described herein below with reference to the figures wherein:

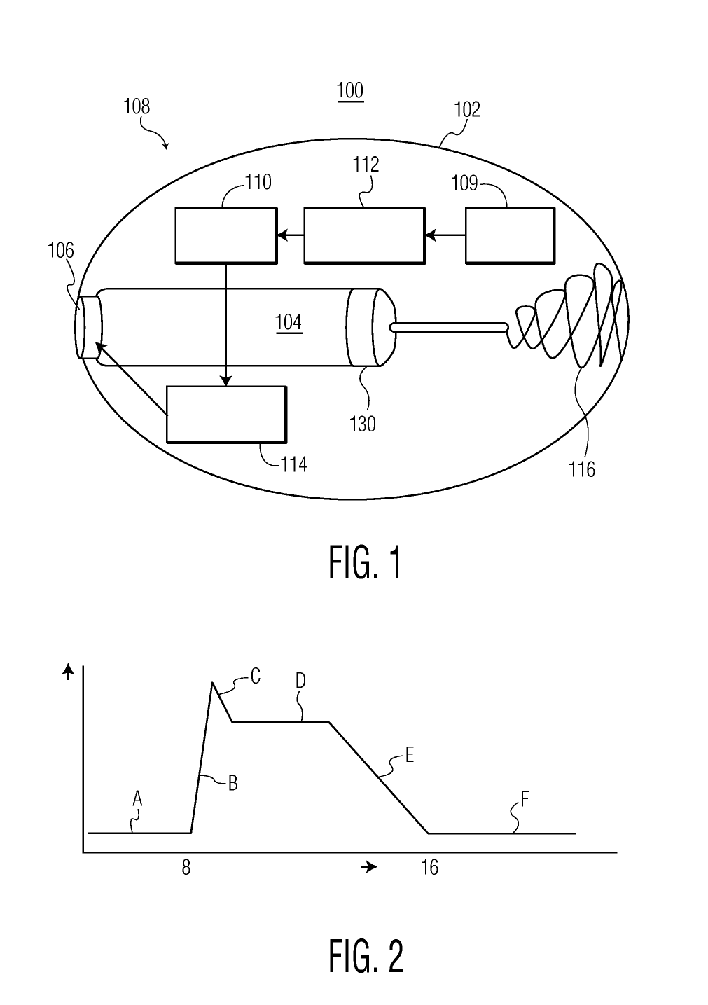

FIG. 1 is a schematic diagram of an electronically controlled capsule in accordance with the present disclosure;

FIG. 2 is a chart illustrating an exemplary preset dispensing timing pattern for the electronically controlled capsule in accordance with the present disclosure;

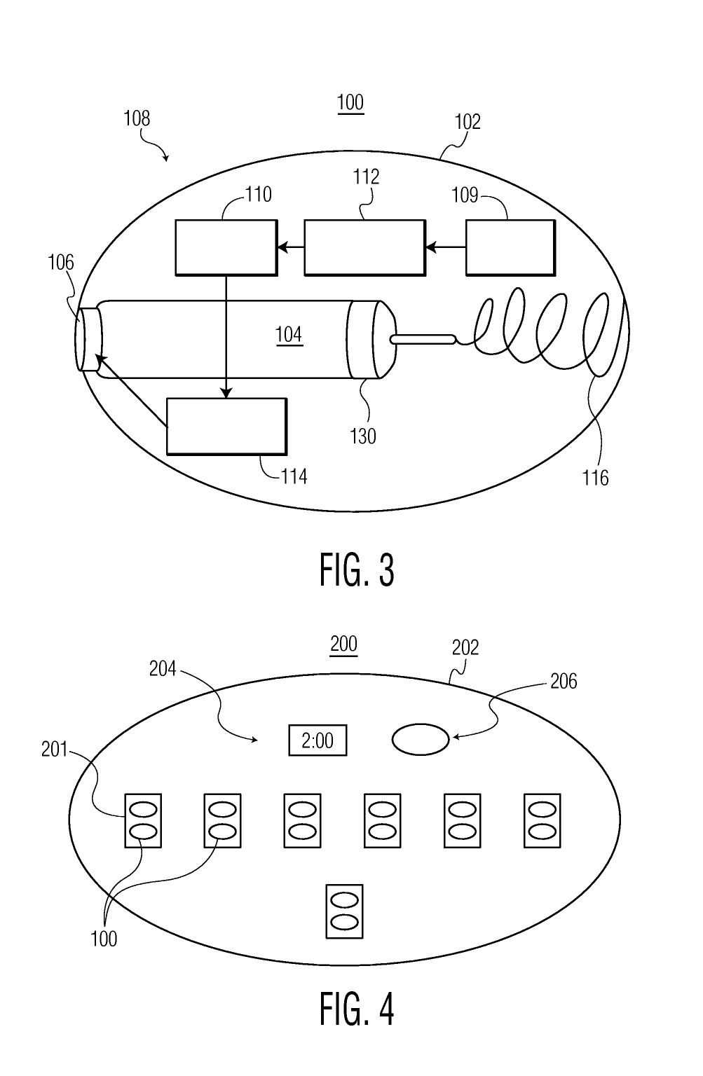

FIG. 3 is a schematic diagram of the electronically controlled capsule dispensing a medicament in accordance with the present disclosure;

FIG. 4 is a diagram of a kit having a plurality of electronically controlled pills tailored for administration to a particular individual;

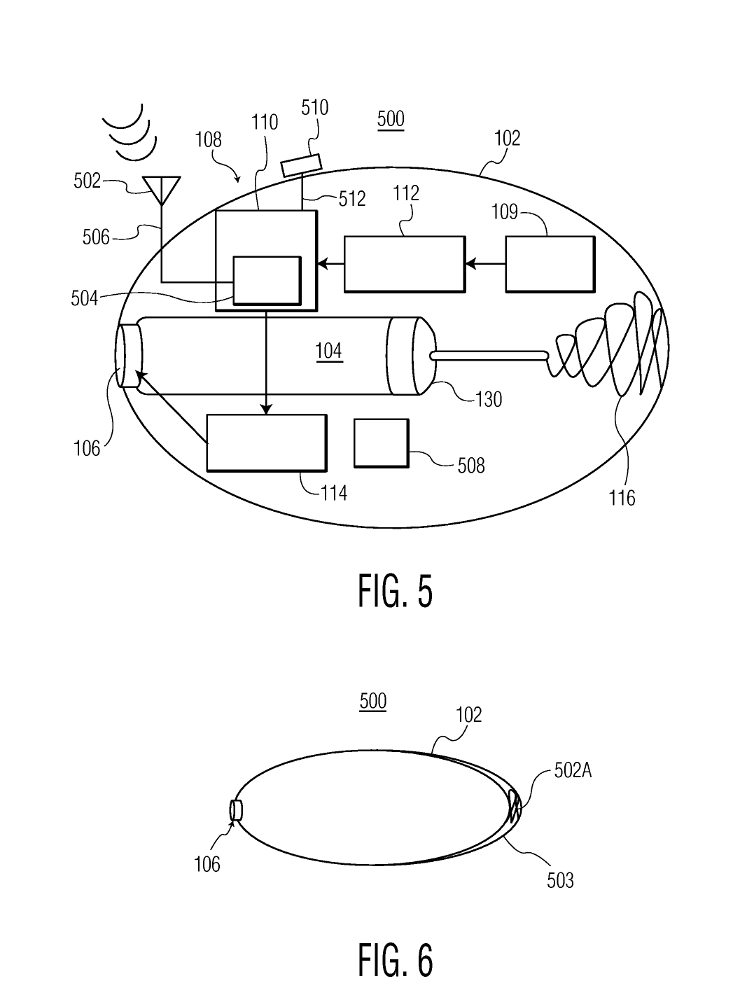

FIG. 5 is a schematic diagram of a remote-controlled pill in accordance with a first embodiment of the present disclosure;

FIG. 6 is a schematic diagram of a remote-controlled pill in accordance with a second embodiment of the present disclosure;

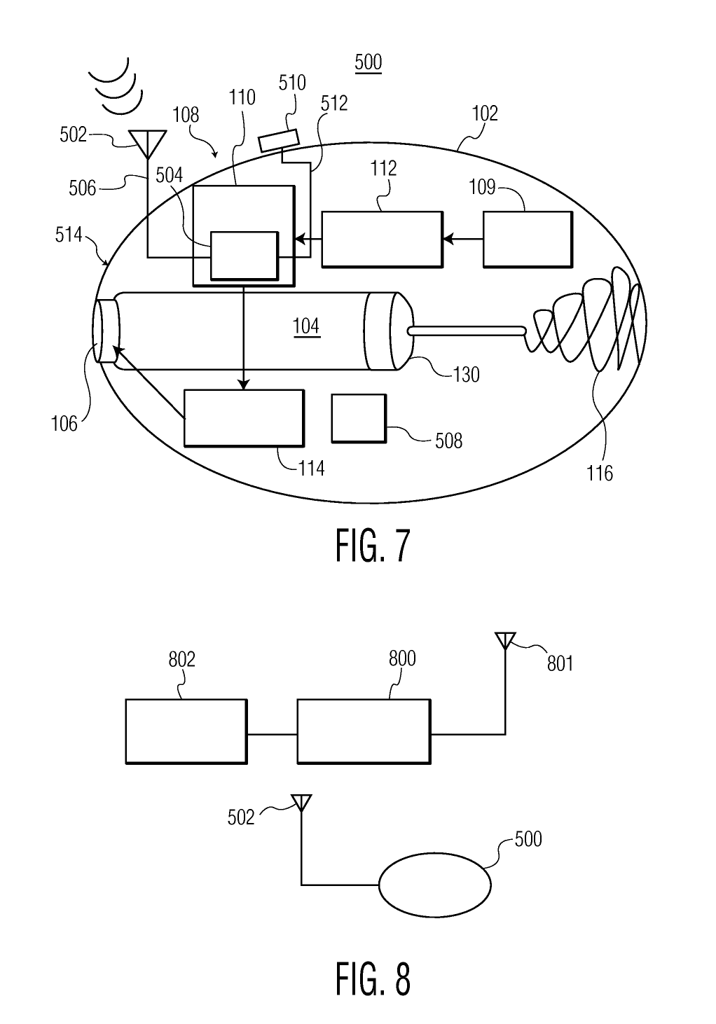

FIG. 7 is a schematic diagram of a remote-controlled pill in accordance with a third embodiment of the present disclosure;

FIG. 8 is a block diagram of a dose managing system for controlling dispensing of a medicament by a remote-controlled pill in accordance with the present disclosure;

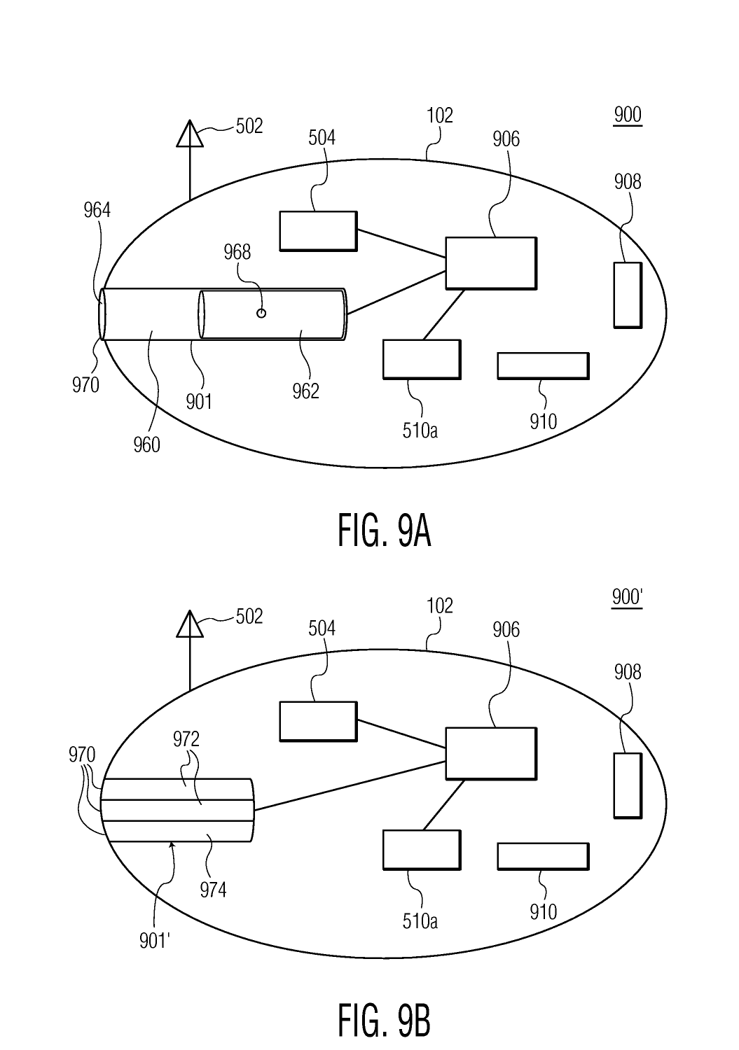

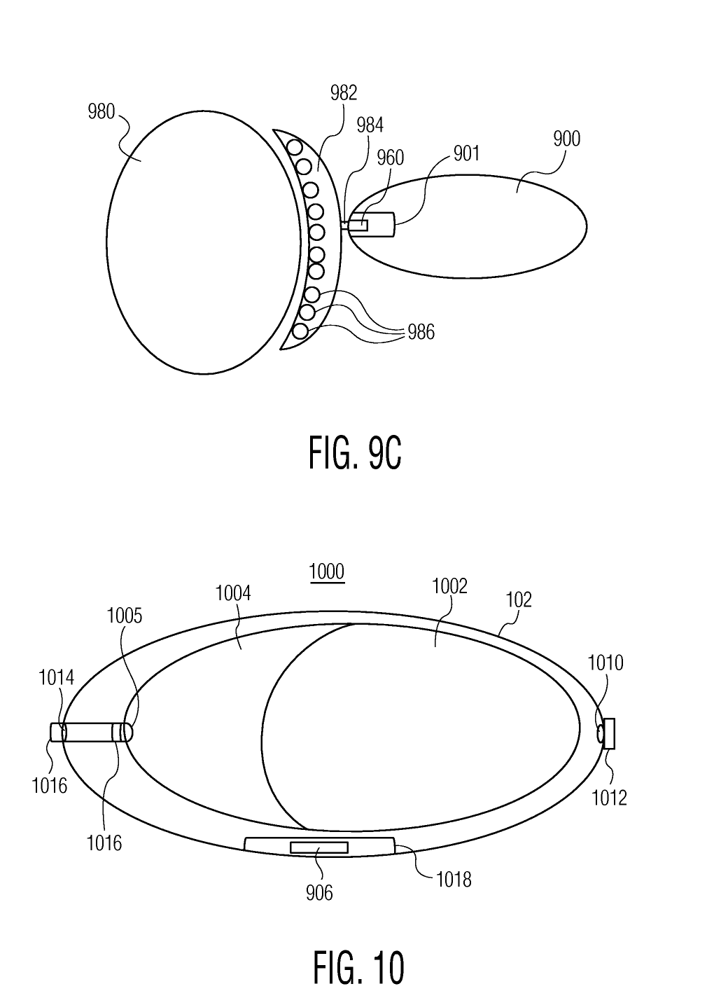

FIG. 9A is a schematic diagram of an electronically controlled capsule for dispensing medicament in accordance with another embodiment of the disclosure;

FIG. 9B is a schematic diagram of an electronically controlled capsule for dispensing medicament in accordance with still another embodiment of the disclosure;

FIG. 9C is a schematic diagram of a medicament dispensing system of an electronically controlled capsule in accordance with an embodiment of the disclosure;

FIG. 10 is a schematic diagram of an electronically controlled capsule for dispensing medicament having a controlled osmotic pressure mechanism in accordance with an embodiment of the disclosure;

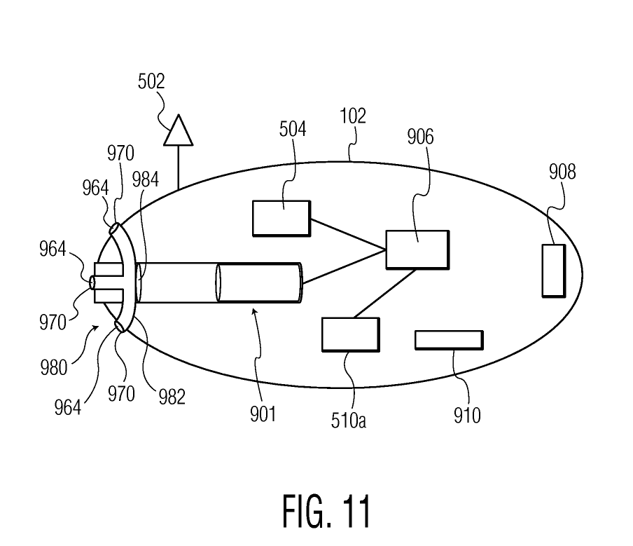

FIG. 11 is a schematic diagram of an electronically controlled capsule having multiple apertures for dispensing medicament in different directions in accordance with another embodiment of the disclosure;

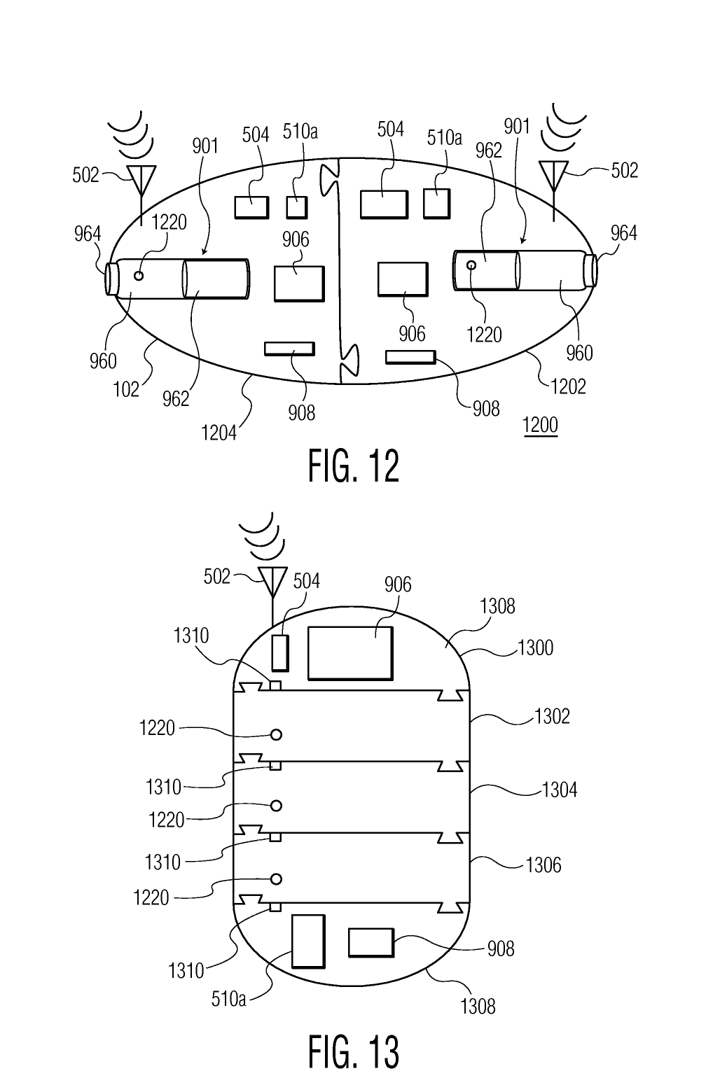

FIGS. 12 and 13 are schematic diagrams of an electronically controlled capsule for dispensing medicament having a modular configuration in accordance with different embodiments of the disclosure;

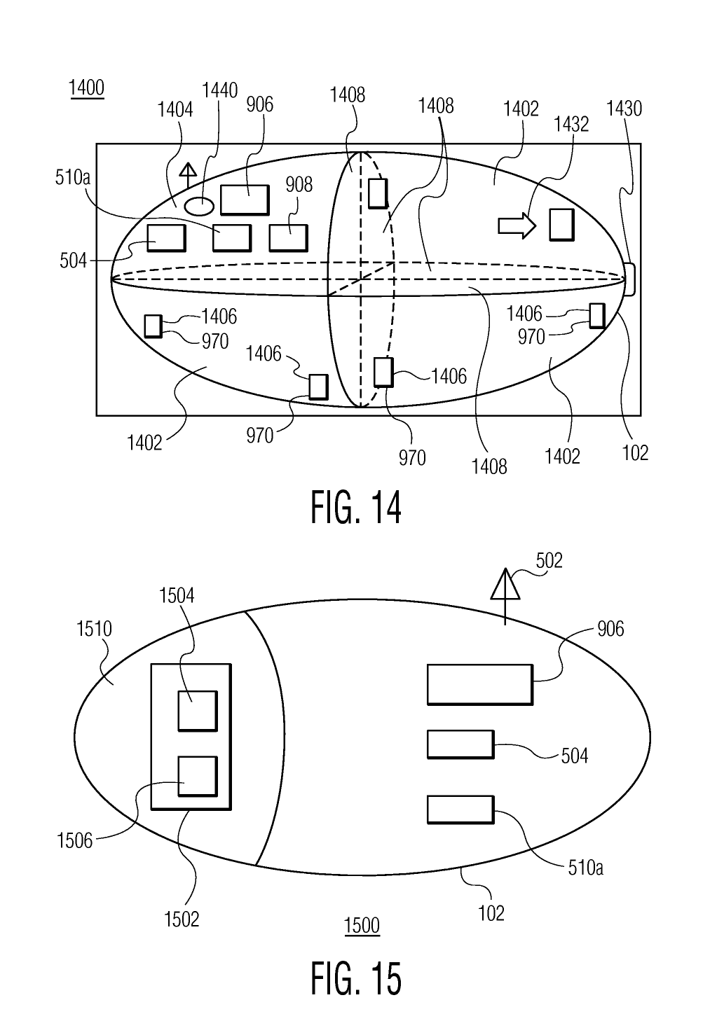

FIG. 14 is a schematic diagram of an electronically controlled capsule for sampling body fluids in accordance with the present disclosure;

FIG. 15 is a schematic diagram of an electronically controlled capsule for sensing visual marks deposited in the alimentary tract in accordance with the present disclosure;

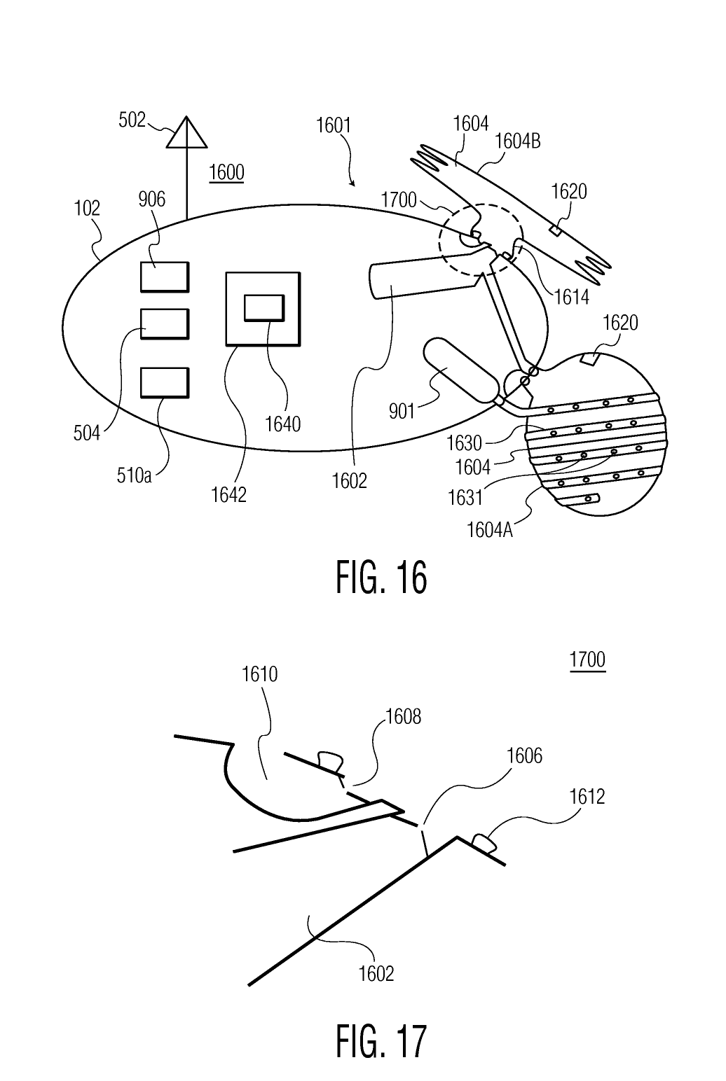

FIG. 16 is a schematic diagram of an electronically controlled capsule having a braking system in accordance with the present disclosure;

FIG. 17 is an enlarged schematic diagram of a pressurizing valve, depressurizing valve and exhaust channel area of one air bag of the capsule shown in FIG. 16;

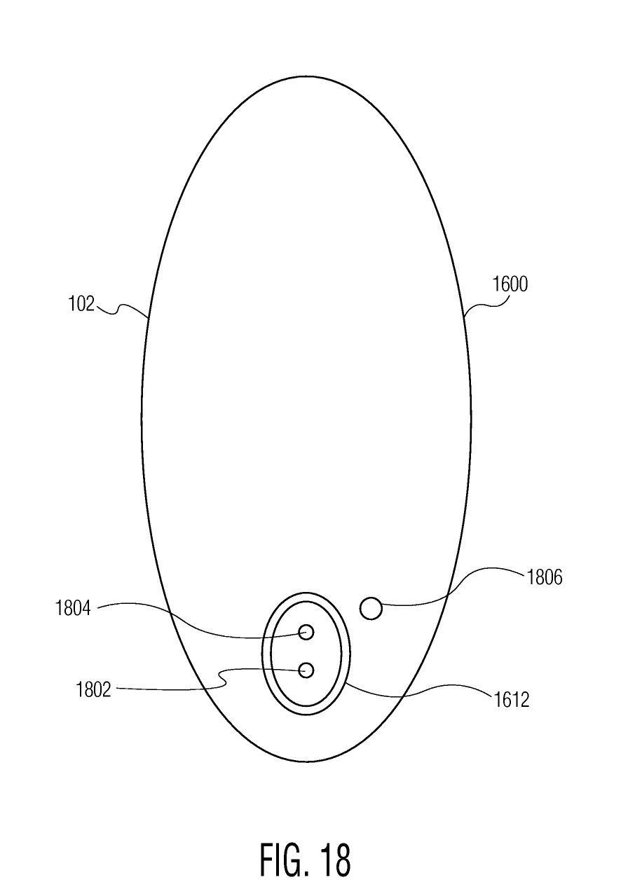

FIG. 18 is a schematic diagram of a top view of the capsule shown in FIG. 16;

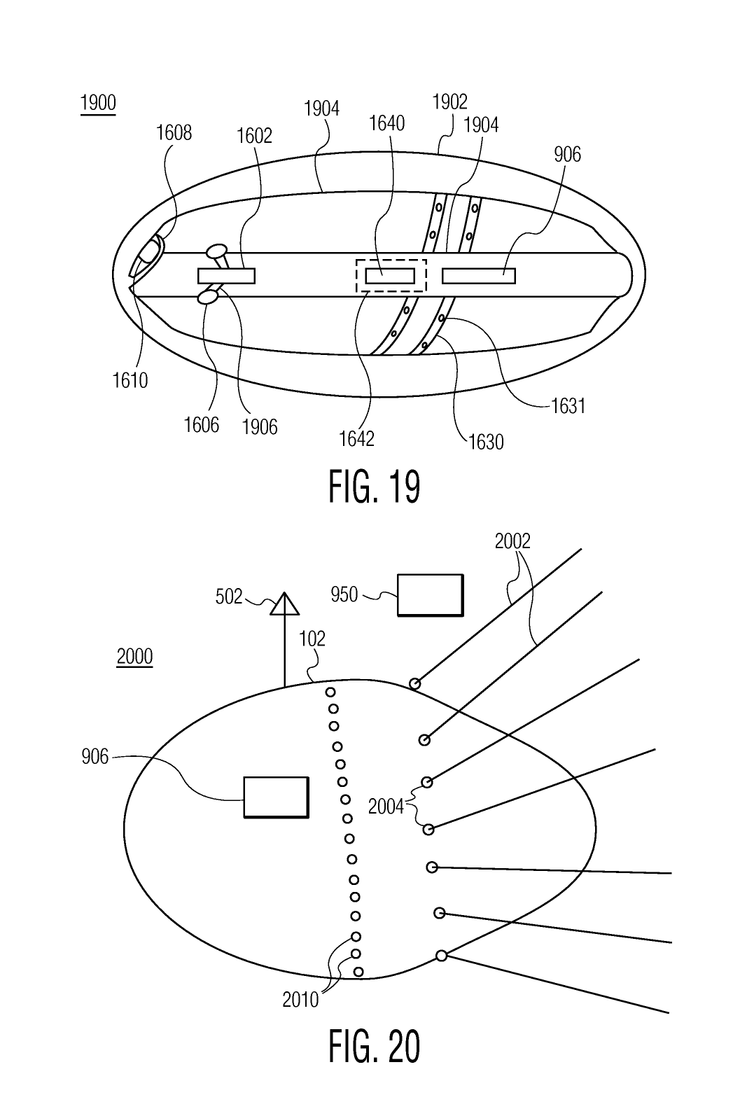

FIG. 19 is schematic diagram of an electronically controlled capsule having a braking system in accordance with another embodiment of the disclosure;

FIG. 20 is a schematic diagram of a capsule for generating a topographical mapping of a traversed alimentary tract;

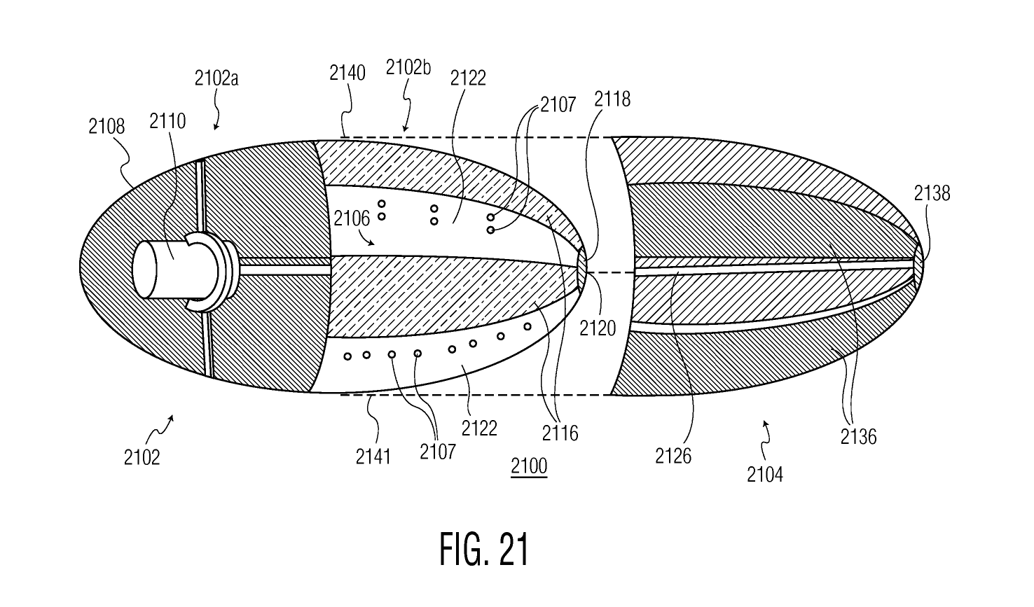

FIG. 21 is an exploded perspective view with parts separated of another embodiment of an electronically controlled capsule for administering radiation in accordance with another embodiment of the disclosure;

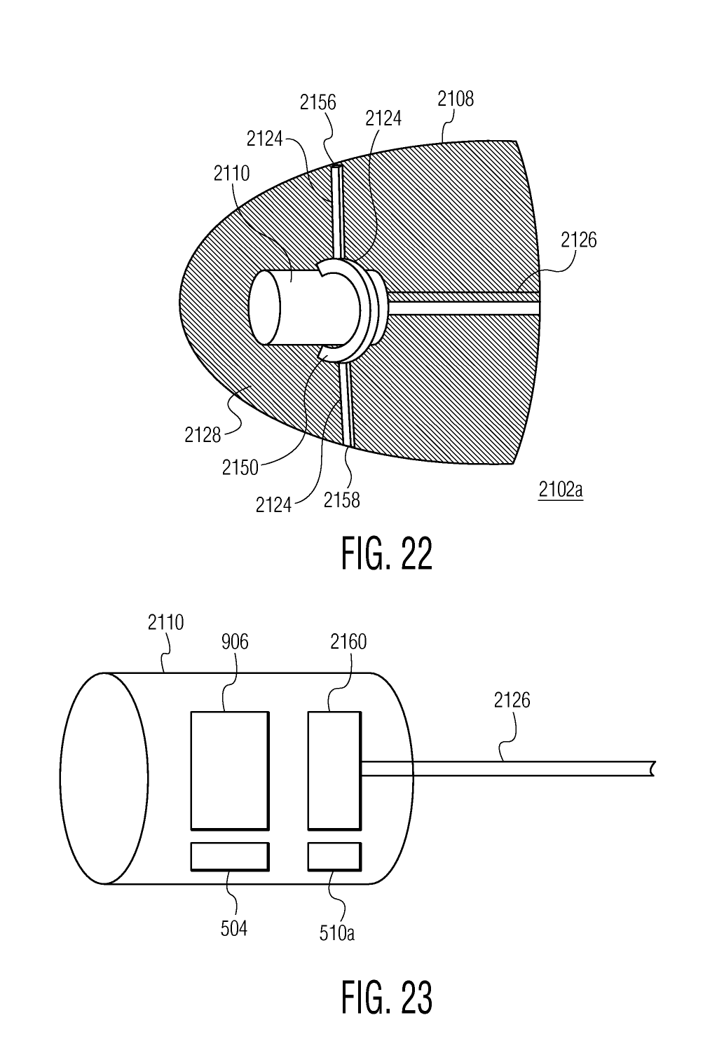

FIG. 22 is a cross-sectional side perspective view of a portion of the capsule shown in FIG. 21;

FIG. 23 is a block diagram of a portion of the capsule housed within a control housing of the capsule shown in FIG. 21;

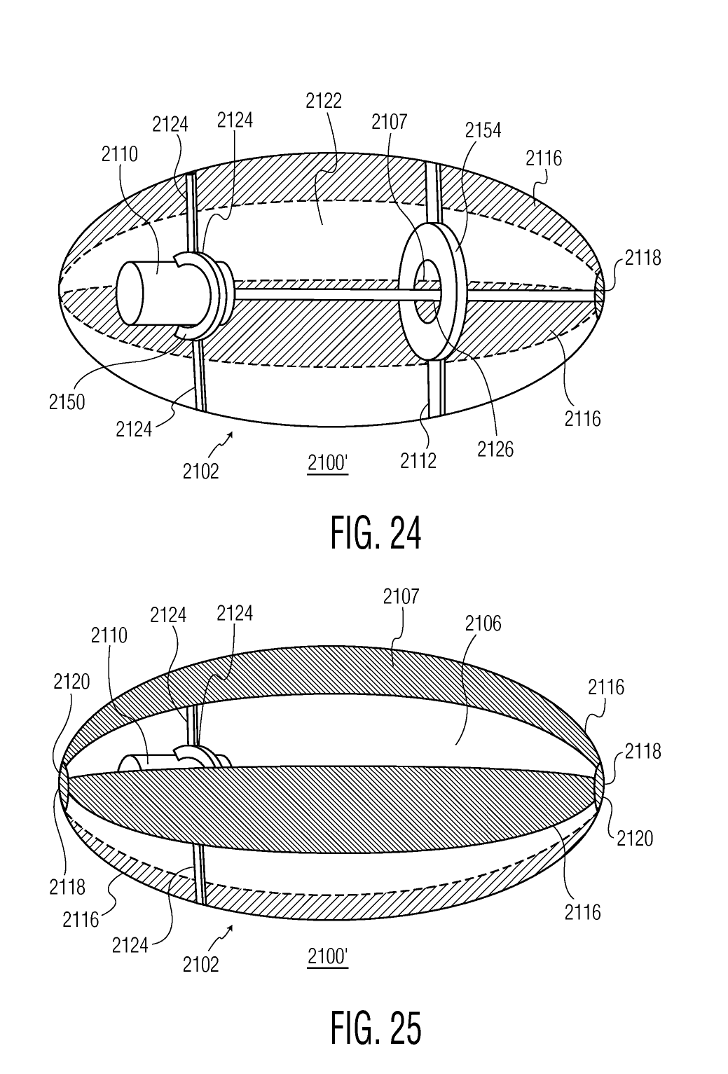

FIG. 24 is a cross-sectional side perspective view of a main body portion of a capsule in accordance with another embodiment of the capsule shown in FIG. 22;

FIG. 25 is a side perspective view of a main body of a capsule in accordance with another embodiment of the capsule shown in FIG. 21;

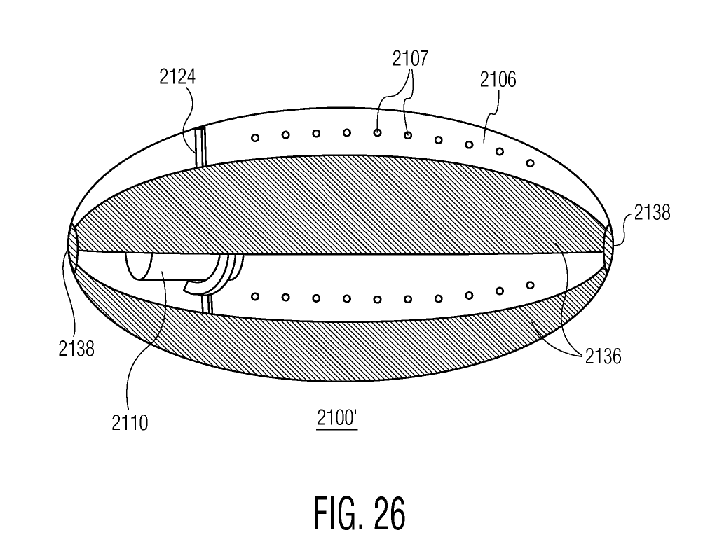

FIG. 26 is a perspective view of the main body of the capsule shown in FIG. 25 assembled with an adjustable module of the capsule;

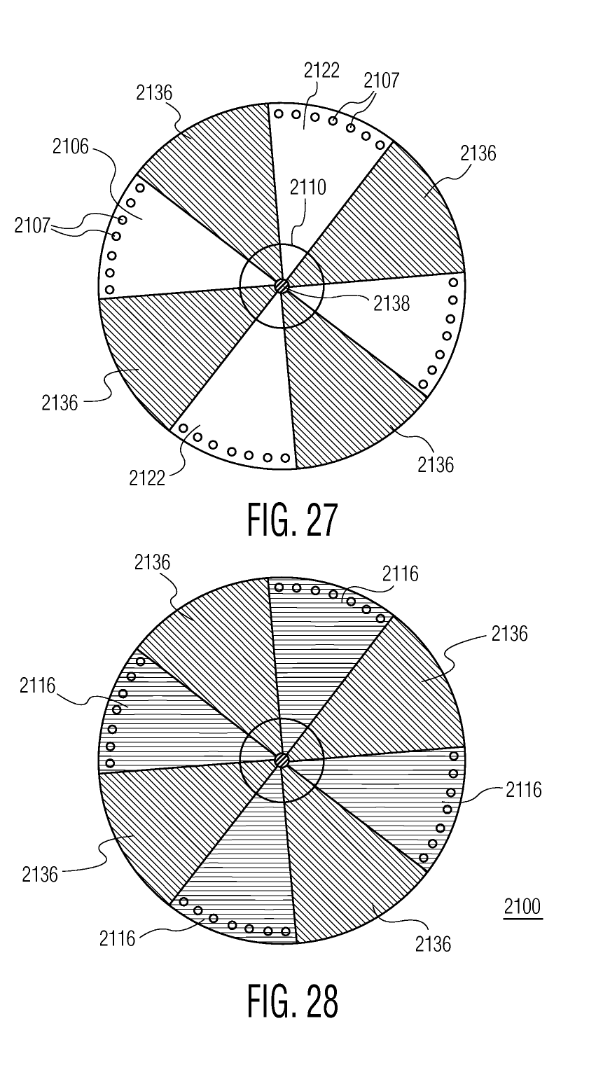

FIG. 27 is an end view shown in an open position of an assembled capsule in accordance with the embodiments shown in FIGS. 21 and 25; and

FIG. 28 is an end view shown in a closed position of the assembled capsule in accordance with the embodiments shown in FIGS. 21 and 25.

A first exemplary embodiment of an electronically controlled capsule or medicament delivery system according to the present disclosure is shown by FIG. 1, and further described with specificity hereinafter. The electronically controlled capsule 100 is a self-contained, electronically controlled medicine delivery system. As described in detail below, the electronically controlled capsule 100 includes programmed electronics that control a release mechanism according to a dispensing pattern for dispensing a medicament. The capsule 100 is made from bio-compatibles materials such that the capsule 100 is bio-compatible for at least the amount of time it requires to traverse the gastrointestinal tract. The bio-compatible materials are preferably stable in room temperature, such that the capsule has a long shelf life. As used herein and in the claims the word “medicament” refers to medicines, non-medicinal substances, contrast agents, gases, fluids, liquids, chemicals, radiological agents, imaging or medical markers, sensors for monitoring the person's vitals, etc.

The electronically controlled capsule 100 includes an outer shell or housing 102; a medicament reservoir 104 for storing a medicament; an electronically controlled release valve or hatch 106 for dispensing the medicaments stored in the medicament reservoir 104; control and timing circuitry 108 for opening and closing the valve 106; and a battery 109. The control and timing circuitry 108 opens and closes the valve 106 throughout a dispensing time period in accordance with a preset dispensing timing pattern as further described below. The preset dispensing timing pattern is pre-programmed and is not susceptible to a person's physiological processes and conditions, mood, earlier-administered medicaments, etc.

The shell 102 is preferably manufactured from materials used to fabricate implantable devices, including pacemaker leads and cardiac prosthesis devices, such as artificial hearts, heart valves, intraaortic balloons, and ventricular assist devices. These materials include Pellethane® 2363 polyether urethane series of materials available from Dow Chemical Company and Elasthane polyether urethane available from the Polymer Technology Group, Inc. Other materials include PurSil® and CarboSil® also available from the Polymer Technology Group, Inc.

The amount that the valve 106 is opened at each moment in time (e.g., each second) of the dispensing time period is dependent upon the preset dispensing timing pattern which is programmed within timing circuitry 110 of the control and timing circuitry 108. The dispensing time period is defined as the time period from when the electronically controlled capsule 100 is placed in a person's mouth to the time all of the medicament stored within the medicament reservoir 104 has been dispensed, or the day (24-hour period) has expired. This 24-hour period may be shifted slightly to account for differences in absorption in the stomach versus the colon.

As shown by the exemplary preset dispensing timing pattern illustrated by FIG. 2, at dispensing time periods A, D and F, identical quantities of the medicament are dispensed throughout each of these dispensing time periods. Therefore, during these dispensing time periods, the valve 106 is kept open by the control and timing circuitry 108 to provide a fixed valve opening (or frequency of opening) for dispensing a predictable quantity of the medicament at each moment in time of dispensing time periods A, D and F. Approximately the same amount of medicament is dispensed at each moment in time during dispensing time periods A and F. During dispensing time period D, a higher quantity of medicament is dispensed than during dispensing time periods A and F.

However, at dispensing time periods B, C and E, as shown by FIG. 2, different quantities of the medicament are dispensed at each moment in time. Therefore, during dispensing time periods B, C and E, the valve opening is varied accordingly by the control and timing circuitry 108 to dispense a quantity of the medicament varying at each moment in time. During dispensing time period B, the quantity of medicament dispensed during each moment in time is increased compared to the previous moment in time; whereas during dispensing time periods C and E, the quantity of medicament dispensed during each moment in time is decreased compared to the previous moment in time.

In accordance with the present disclosure, during the entire dispensing time period, the control and timing circuitry 108 is programmed for closing the valve 106 and controlling the amount the valve 106 is opened for controlling the size of the valve opening. By controlling the size of the valve opening or frequency of valve opening, such as is enabled by micro fluidic systems of inkjet printers and the like, the electronically controlled capsule 100 can precisely control the quantity of medicament released during each moment in time (e.g., each second) of the dispensing time period.

By knowing the quantity or approximate quantity of medicament released during each moment in time by referring to a time release pattern, such as the one shown by FIG. 2, one can precisely determine the cumulative amount of medication released over a particular time period of the dispensing time period. For example, one can determine the cumulative amount of medicament released during the first six hours of the dispensing time period, the first two hours until the last hour of the dispensing time period, the entire dispensing time period, etc. One can also determine the amount of medicament dispensed during a particular moment of the dispensing time period, such as at two hours and fifteen minutes after the capsule 100 has been administered.

The preset dispensing timing pattern may be varied from one electronically controlled capsule 100 to another by programming the control and timing circuitry 108 of each capsule 100 to have a different preset dispensing timing pattern. Therefore, two individuals can be administered the same medicament utilizing two different preset dispensing timing patterns. The timing patterns can be determined using a look-up table which correlates one or more characteristics of a person with one or more preset dispensing timing patterns.

For example, a look-up table can correlate at least one of age, gender, weight, etc. with preset dispensing timing patterns. The person would then be administered an electronic capsule 100 which is programmed with one of the determined preset dispensing timing patterns. Accordingly, the capsule 100 of the present disclosure enables the same medicament to be administered to different individuals using different dispensing timing patterns.

Additionally, for young and old people that have difficulty taking or remembering to take capsules, the preset dispensing timing patterns are a way to reduce the number of capsules taking during a particular time period, e.g., a 24-hour period. All of the medicament required to be administered during the particular time period to an individual can be provided in one capsule 100 having a preset dispensing timing pattern for dispensing the medicament according to predetermined quantities during the particular time period. If the payload in one capsule is insufficient, then two electronically controlled capsules are used to dispense the same medicament, where one capsule does not start dispensing the medicament until the other capsule has dispensed its medicament, i.e., its dispensing time period has lapsed or ended. Further, the present disclosure reduces the amount of labor required to administer capsules in places like hospitals, nursing homes and veterinary facilities. By reducing the number of times that capsules are administered, the number of medicament administration errors can also be reduced.

With reference to FIG. 1, the control and timing circuitry 108 includes timing circuitry 110 programmed with the preset dispensing timing pattern, a start timer mechanism 112, a release controller 114 and a pressure mechanism 116. The start timer mechanism 112 enables activation of the timing circuitry 110. The battery 109 powers the control and timing circuitry 108 in order for each of the electromechanical components to operate during the dispensing time period.

In a preferred embodiment, the start timer mechanism 112 is a micro-electromechanical (MEM) mechanism having a sensor 118 for sensing the presence of a liquid, such as water, saliva, etc. When the capsule 100 is taken or administered, the sensor 118 senses the presence of a liquid, and transmits an electrical signal to the timing circuitry 110. In an alternate embodiment the start timer mechanism is a button which is pushed to transmit the electrical signal to the timing circuitry 110. The button is pushed just before the capsule 100 is administered to a person or animal.

In another embodiment, this can be achieved by dissolving a thin, water soluble coating that separates two electrical contacts, enabling the switch to close the circuit. In still another embodiment, the switch is manually triggered by the patient or caregiver.

Upon receiving the electrical signal, the timing circuitry 110 begins to clock the dispensing time period and control the release controller 114 by transmitting a signal thereto. The timing circuitry 110 includes a microprocessor programmed with the preset dispensing timing pattern for relaying the signal to the release controller 114, such that the medicament is dispensed during the dispensing time period substantially according to the preset dispensing timing pattern, such as the one shown by FIG. 2.

The voltage level of the signal relays the size of the valve opening for controlling the quantity of the medicament dispensed at each moment of the dispensing time period substantially according to the preset dispensing timing pattern as shown by FIG. 2. In an alternate embodiment, the signal transmitted by the timing circuitry 110 to the release controller 114 only relays the opening and closing of the valve 106 and not the size of the valve opening.

The release controller 114 is preferably a micro-electromechanical mechanism capable of receiving the signal from the timing circuitry and generating a signal having a variable voltage level to the electronically controlled valve 106 for closing the valve 106 and controlling the size of the valve opening or degree of opening of the valve 106 (in accordance with the voltage level of the received signal). In the simplest case, the release controller 114 is a transistor or D/A circuit that provides voltages to the valve 106 causing it to open or close.

The electronically controlled valve 106 is preferably a micro-electromechanical mechanism capable of being electrically controlled by a signal having a variable voltage levels. Each voltage level corresponds to a different size opening for the valve opening and one voltage level (or no voltage at all, i.e., no signal) corresponds to the valve 106 being closed. The valve 106 is similar in operation to valves used in ink-jet printers for dispensing ink in accordance with the amount that the valve is opened. The valve 106 is characterized as a microfluidic valve for controlling the movement of minute amount of liquids or gases in a miniaturized system.

In an alternate embodiment, the reservoir 104 is a micro-syringe, whereby pressure applied to a plunger of the syringe dispenses the medicament via a needle tip of the micro-syringe which is in fluid communication with an opening in the shell 102. In this embodiment, the opening replaces the valve 106. It is contemplated, however, that a check valve is placed at the needle tip of the micro-syringe to avoid leakage of the medicament during time periods within the dispensing time period where there should be no dispensing according to the preset dispensing timing pattern, and/or for controlling the quantity of medicament dispensed during the dispensing time period.

The pressure mechanism 116 is located outside the medicament reservoir 104 ensuring that the medicament is directed toward the valve 106. In the simplest case, the pressure mechanism 116 is preferably a biodegradable spring as shown by FIGS. 1 and 3. The pressure mechanism 116 can also be another type of spring, a piston, or any mechanism for performing the function of the pressure mechanism 116. That is, for performing the function of applying pressure to a piston-type member 130 when the valve 106 is open to push the piston-type member 130 towards the valve 106. As the piston-type member 130 moves towards the valve 106 pressure within the reservoir 104 causes the medicament to be dispensed as shown by FIG. 3.

In an alternate embodiment, the medicament reservoir 104 is kept under pressure to assure a proper quantity of medicament is dispensed in accordance with the degree of openness of the valve 106, without the need for the pressure mechanism 116. The pressure can be monitored by a pressure sensor which relays the monitored pressure to the control and timing circuitry 108. If the pressure is outside a predetermined range, the circuitry 108 can then adjust the valve opening to increase or decrease the pressure. Naturally, the pressure of the reservoir 104 can be different for each medicament and can depend on the medicament's viscosity.

It is contemplated that a look-up table or other data structure can be assessed by the circuitry 108 which correlates pressure, degree of valve opening, and other parameters, such as period of time in the dispensing time period, for determining, for example, the degree of valve opening by knowing the pressure, and vice versa. Based on the information obtained by assessing the look-up table, the circuitry 108 can then adjust the pressure, the valve opening, etc. These adjustments can be made in order to substantially track the preset dispensing timing pattern programmed within the capsule 100.

According to the present disclosure, all of the medicaments required to be taken during a particular time period, for example, during a 24-hour period, can be provided within one or more electronically controlled capsules 100 which can all be taken at the same time. As such, a treatment system of the present disclosure provides for two or more medicaments to be administered at the same time via the one or more electronically controlled capsules 100. Each capsule 100 has an independent, preset dispensing timing pattern in order to dispense its medicaments within the body according to a dispensing pattern. The dispensing pattern can be varied from person to person depending on each person's physical condition, age, gender, ailments, etc.

The treatment system of the present disclosure enables an individual to take all of his medicaments at substantially the same time, e.g., in the morning or in the evening, and not at different times during a particular time period (e.g., a 24-hour period). The treatment system of the present disclosure further enables a caregiver to administer once per day (i.e., once per a 24-hour period) all of the medicaments for each patient of a hospital or resident of a nursing home (or animals in a shelter or veterinary facility). The system of the present disclosure therefore avoids the need for a caregiver to wake up or otherwise disturb a patient or resident for the sole purpose of administering a medicament, or to track down a patient or resident who may be in a different part of the hospital or nursing home for the sole purpose of administering a medicament.

The present disclosure also provides a kit 200 as shown by FIG. 4 having two or more electronically controlled capsules 100 packaged within a container 202. Each capsule 100 is placed within an indenture or recess 201 of the container 202 and each capsule 100 has an independent, preset dispensing timing pattern programmed therein. The capsules 100 of the kit 200 are custom tailored for an individual (or animal), such that the individual or his caregiver can be provided with the container 202 by a physician, pharmacist, etc.

A timing schedule 204 is provided inside the container indicating when each of the capsules 100 of the kit 200 is to be taken, e.g., the time and day of the week. The timing schedule 204 includes an area 206 where a physician, pharmacist, etc. can write the time when the capsules 100 for each particular day are to be taken, and circle am or pm. Two or more capsules 100 may need to be taken at a particular time of a given day, as shown by FIG. 4, where each capsule has a different medicament stored therein and a different preset dispensing timing pattern. As such, an individual can take all of the capsules 100 which are indicated to be taken at the particular time of the given day and not take any other capsules 100 until the same time the following day.

Since each of the capsules 100 of the kit 200 has a programmed preset dispensing timing pattern, there is little or no concern that the medicaments from each capsule 100 would interact with each other even though the capsules 100 are taken at the same time. For example, one of the capsules 100 of the kit 200 can start dispensing immediately, while another capsule 100 of the kit 200 would not start dispensing until three hours later.

In an alternate embodiment of the capsule 100, as shown by FIG. 5, and designated generally by reference numeral 500, the remote-controlled capsule 500 is provided with an antenna 502 for receiving control signals, such as RF control signals, for remotely communicating commands or instructions to the capsule 500 for controlling the capsule 500. The antenna 502 may also transmit information from the capsule 500 to the outside as further described below. In an alternative embodiment, as shown by FIG. 6, an antenna 502A can be provided in a folded configuration and encapsulated by a soluble membrane 503. When the capsule 500 is ingested, the soluble membrane 503 is dissolved, which then allows the antenna 502A to unfold.

The capsule 500 operates substantially in the same manner as the capsule 100, except for the operational differences described below with respect to the former capsule's remote-control capabilities. The capsule 500 includes the same components as the capsule 100 where identical reference numbers in FIGS. 1 and 5 identify similar components. A plurality of capsules 500 can be packaged as a kit as described above with reference to FIG. 4.

The control signals received by the capsule 500 are transmitted to RF communication circuitry 504 within the timing circuitry 110 via wire leads 506. The RF communication circuitry 504 includes a receiver and processing circuitry for processing and analyzing the received RF control signals and accordingly determining one or more particular actions indicative of the instructions or codes provided by the control signals. The actions are determined by correlating the instructions or codes with one or more actions using a data structure, such as a look-up table, within the timing circuitry 110.

The instructions provided by the control signals can include overriding the preset dispensing timing pattern programmed within the timing circuitry 110 for one or more moments in time during the dispensing time period. This may be necessary to dynamically increase or decrease the amount of medicament being dispensed during a particular time during the dispensing time period due to the person's vitals at a particular moment in time and other factors. The person's vitals can be monitored using conventional systems and sensors. One or more of these sensors can be provided within the capsule 500 itself for sensing the person's vitals as the capsule 500 traverses the gastrointestinal tract and for transmitting the information to the timing circuitry 110 which in turn dynamically adjusts the dosage based on the person's sensed vitals.

The instructions provided by the control signals can further change the dispensing timing pattern by reprogramming the timing circuitry 110 with a different dispensing timing pattern. The control signals can further provide instructions as to which moment in time of the new dispensing timing pattern the dispensing of the medicament should commence. The new dispensing timing pattern can be transmitted via the control signals or be stored within a memory of the timing circuitry 110, where the memory includes a plurality of dispensing timing patterns and the control signals indicate which dispensing timing pattern is desired.

The control signals can also instruct the control and timing circuitry 108 to terminate the dispensing of the medicament within the body, in case the wrong medicament was administered, the wrong dose was prescribed, the person had an adverse reaction to the medicament, etc. The control signals can further instruct the control and timing circuitry 108 to release a bowel slowing medication, such as Lomotil®, stored within a reservoir or micro-sac 514 (FIG. 7) of the capsule 500 for temporarily halting the progress of the capsule 500 through the gastrointestinal tract. The bowel slowing medication can be released in tandem with the medicament stored within the reservoir 104. The bowel slowing medication can also be provided within a separate capsule.

The generation and transmission of the control signals can be synchronized with an external system, such as an MRI system, ultrasound imaging system, etc., for dispensing the medicament in accordance with the person's vitals monitored by the external system, the mode of operation of the external system, etc. The medicament can be an oral contrast agent used to enhance diagnostic images. An example of such a contrast agent is Gastromark® for MRI images and Barium for CT images.

In addition to releasing contrast agents for each modality, the release time can be used for diagnostic purposes. A common problem in multi-modal imaging (e.g., any combination of CT, PET, MRI, Ultrasound, X-Ray, etc.) is the registration of images. Between images, patient motion causes difficulties in ‘registering’ different images to one another. Patient motion includes walking between the exams as well as voluntary and involuntary internal motions such as breathing, heart beating, and digestion.

The capsule 500 can be used to release contrast agents in particular areas that can be estimated by time in order to minimize the contrast agent required or concentrate it in a particular area. Use of contrast agent does not only register the images in terms of location, but in terms of time, and even across multimodalities. This fourth dimension can improve the accuracy of co-registration, even using multimodalities.

The controlled timing of contrast agents can also be used diagnostically to measure the timing through different parts of the alimentary tract. This demonstrates the effectiveness of peristaltic action (the movement of muscles that propel food through the alimentary tract). Locating failed areas of peristaltic action can aid in the diagnosis of diseases, such as Crohn's disease and other obstructive bowel problems.

The control signals preferably transmit unique identification information which is used by the timing circuitry 110 to ensure that the received control signals are for the respective capsule 500. This prevents control signals from initiating an action to a capsule 500 other than the intended capsule 500. The identification information can be a unique serial number which is programmed within the timing circuitry 110. If the received serial number does not match the programmed serial number, the timing circuitry 110 does not respond to the received control signals. Accordingly, the timing circuitry 110 does not perform any action, such as the actions described above.

The communication circuitry 504 includes a transmitter for transmitting signals from the capsule 500. The signals are generated by the communication circuitry 504 for providing information to a caregiver or the person. Information that can be provided includes the particular moment in time of the dispensing time period; the cumulative quantity of medicament dispensed from the beginning of the dispensing time period to a particular moment in time of the dispensing time period; the average quantity of medicament dispensed during each moment in time of the dispensing time period (e.g., each second); etc.

Additionally, the transmitter can provide a signal for alerting or notifying a caregiver or the person that the capsule 500 has been taken, in case the caregiver or the person do not remember if the capsule 500 was or was not taken. The transmitter can also provide a signal if the capsule 500 after diagnostic tests are executed by the control and timing circuitry 108 and it is determined that the capsule 500 has malfunctioned, in cases such as if the capsule 500 is not dispensing the medicament, the medicament is not being dispensed according to the preset dispensing timing pattern, etc.

The capsule 500 includes an optional RFID tag 508 for tracking, identification, inventory and other purposes using an RFID reading system. The RFID tag 508 can also be used to determine if the capsule 500 was administered by a caregiver or taken by the person, and if so, the RFID tag 508 can be used to determine the general location of the capsule 500 within the gastrointestinal tract.

The capsule 500 further includes a piezo-electric element and associated circuitry 510 for remotely transmitting commands via the communication circuitry 504 to the timing circuitry 110 for remotely controlling the capsule 500. The element 510 is preferably affixed to the housing 102 and is capable of being vibrated at one or more predetermined frequencies. The vibration is caused by placing an ultrasound probe, hydrophone or other vibration-causing device in proximity to the person.

The frequencies caused by the element 510 are converted to electrical signals by the associated circuitry. The electrical signals are transmitted to the timing circuitry 110 via wire lead 512 where they are processed for determining an action to perform. The action can be one of the actions described above with reference to the control signals provided to the timing circuitry 110 via the wire leads 506. The action is preferably determined by correlating the vibration of the element 510 to an action using a data structure, such as a look-up table, stored within the control and timing circuitry 108 and accessible by the timing circuitry 110.

With reference to FIG. 8, the communication circuitry 504 of the remote-controlled capsule 500 is able to communicate with a transmitter/receiver 800 via antenna 502 (or piezo-electric equivalent 510) of a dosage management system 900. The transmitter/receiver 800 forwards commands determined by a Dose Manager 802 via an antenna 801. The Dose Manager 802 is a computing device, such as a personal computer, which may be connected to the Internet or other network, such as a LAN. The Dose Manager 802 receives patient vital sign information electronically from advanced monitoring systems and/or biosensor devices including pulse, oxygen level from a pulse-oximeter, EKG, blood pressure, blood protein level, body temperature, body fluid composition; and/or from a manual computer entry, such as from a keyboard. Based on the received information, the dosage of the medicament is adjusted as described below.

The biosensor devices may include electrodes positioned on the user. One or more biosensor devices can be included within the capsule 500 itself. The patient or doctor may also enter auxiliary information into the Dose Manager 802, such as the degree or level of pain, which typically cannot be measured directly.

The information received by the Dose Manager 802 is used by the control and timing circuitry 110 to automatically control the desired dosage or the quantity of medicament to be dispensed by the remote-controlled capsule 500. External or non-measured information can also be used to direct the desired dosage. For example, a barometric reading, and weather reported or anticipated (snow, rain, etc.) for a particular zip code (such as is available on www.weather.com) may drive the amount of arthritis medication delivered by the remote-controlled capsule 500. Similarly, pollen counts and other allergens are often available via the Internet for particular areas. Allergy medication can be dispensed as a function of the particular allergen sensitivity of the patient. For more accurate and automatic control, a GPS located on the patient can send information to the Dose Manager 802 to determine the current location and zip code of the patient. Wireless communication, such as by cell phone can alternatively substitute for the Internet or communication between the GPS and Dose Manager 802.

Information derived from a patient's electronic calendar or schedule stored in a PDA, or alarm clock can also be used to infer proper dosing. For example, an early appointment may trigger earlier release of arthritis medication, enabling the patient to wake and become more productive as a function of the demands of the day.

With reference to FIGS. 9A and 9B, a capsule 900 in accordance with a further embodiment of the present disclosure is shown. The capsule 900 is a free standing capsule which is not attached structurally to a device located external to the patient. Exemplary capsule 900 includes housing 102, medicament dispensing system 901 for dispensing a medicament, a MEMS sensor module 902 including at least one sensor 904, control circuitry 906, a power source 908, an optional identification tag 910, such as an RFID tag, and/or a communication assembly. The communication assembly includes antenna 502 (which is optionally collapsible), ultrasound transducer element and associated circuitry 510a and/or communication circuitry 504. Communication circuitry 504 is preferably included in control circuitry 906 or in communication with control circuitry 906 for interfacing between the antenna 502 and the control circuitry 906 and/or between the piezo-electric element 510a and the control circuitry 906.

Control circuitry 906 may send/receive control signals via the communication assembly from remote devices, such as the remote processing device 950 or another capsule, such as a capsule 900 or other capsule having communication and processing capabilities. The control signals may include information for identifying the target recipient, e.g., addressing the recipient. Each capsule 900 preferably has a particular identification number or address assigned to the capsule 900 in order that the capsule 900 process only control signals addressed to the capsule. The identification number, such as a unique serial number, may be programmed into the control circuitry 906, such as into an ePROM included in the control circuitry 906

Control circuitry 906 is in communication with the medicament delivery system 901 and the sensor module 902 for receiving information and/or sending command signals, such as control signals. Communication between components of the capsule 900 may be wired or wireless, such as via optical signals.

The control circuitry 906 is preferably in communication with a remote processing device 950 via wireless communication. For example, communication between the control circuitry 906 and the remote processing device 950 may be provided via antenna 502 and remote transmitter/receiver device 800. Alternatively, or additionally, communication between the control circuitry 906 and the remote processing device 950 may be provided via element 510a and an external ultrasound probe 952 having a transducer 954.

Element 510a is a transducer element, such as a piezo-electric element, and may be configured operationally similar to element 510 of FIG. 5, however element 510a is preferably capable of two-way communication for transmitting as well as receiving signals. Ultrasound signals transmitted by the element 510 to the remote processing device 950 are preferably transmitted at a low frequency for adequate transmission through the patient's body in order to exit the patient's body. In a preferred embodiment, a protocol based on Zigbee (which is appropriate for low bandwidth communication) is used for communication between the capsule 900 and the remote processing circuitry 950.

It is further envisioned that the control circuitry 906 may communicate with control circuitry of another capsule device internally placed (implanted or ingested) within the patient. Communication may be facilitated through antenna 502 and/or element 510a for capsule-to-capsule communication. Due to proximity between the capsules within the patient's body, a variety of frequencies and protocols may be used. It is further envisioned that a capsule having other components instead of or in addition to components of capsule 900, such as instead of the medicament dispensing system 901 and/or the sensor module 902, may be configured for communication with the remote processing circuitry 950 and/or another capsule. For example, a capsule having a camera may transmit a signal to another capsule behind it, such as to instruct the other capsule to perform an action, e.g., to dispense medication at a particular location sensed or imaged by the capsule having the camera.

The control circuitry 906 includes at least one processing device, such as a microprocessor. The processing device executes at least one software module 980 including a series of programmable instructions which can be stored on a computer-readable storage medium accessible by the microprocessor, such as ROM, flash memory, or transmitted via propagated signals for performing the functions disclosed herein and to achieve a technical effect in accordance with the disclosure. The control circuitry 906 may be programmed by a remote processing device, even when the capsule 900 is located internal to the patient. The microprocessor is not limited to execution of the software module 980 described. The functions of the respective software modules 980 and modules included within the software module 980 may be combined into one module or distributed among a different combination of modules. Preferably, the microprocessor executes the software module 980, processes received signals, such as from the sensor module 902 and/or the remote processing circuitry 950, and generates control signals for controlling components of the capsule 900, such as the medicament dispensing system 901 and/or the sensor module 902. The control circuitry 906 further includes timing circuitry and mechanisms and/or circuitry for starting and/or controlling the timing circuitry, as well as any interfaces for interfacing with other components of the capsule.

It is contemplated that the control circuitry 906 or a portion thereof may be located remote from the capsule 900 and send control signals to the capsule, where the control signals may be digital signals for processing by control circuitry 906 in the capsule 900, or the control signals may be RF or ultrasound signals for controlling components of the capsule 900.

The identification tag 910, such as an RFID tag, provides information to the remote processing circuitry 950 and/or another capsule for identifying the capsule 900, which may include a unique identification and/or identify a classification to which the capsule 900 belongs. The power source 908 includes at least one power source, such as a battery, which provides power to the control circuitry 906 and/or other components of the capsule 900 which need power. An exemplary battery is a thin film lithium battery (e.g., available from Frontedge Technologies™, located in Baldwin Park, Calif.), having a small footprint and a suitable shelf life (e.g., 1% discharge/year). The battery may further be selected from other known batteries, such as photo lithium, silver oxide, lithium coin cells, zinc air cells, alkaline, etc. . . . It is envisioned that the capsule 900 may not include a power source 908 (e.g., a battery), and may use passive power. It is contemplated that the power source 908 include a device configured for scavenging power from another device, which may employ electrostatic, micro fuel cells, micro-heat, temperature gradient, etc.

The remote processing device 950 includes at least one processor, which may include a network of processors, which further may include the dose manager 802, a decision support system (DSS) and/or a knowledge base. The at least one processor of the remote processing device 950 may analyze information, such as information provided by the capsule 900, information provided by additional sensors remote from the capsule 900, and/or information stored in an accessible database for providing real time decision making. Furthermore, the at least one processor of the remote processing device 950 may provide control signals to the control circuitry 906 for controlling operation of components of the capsule 900 in real-time.

It is envisioned that the position of the capsule 900 may be monitored by external means, such as by imaging the patient and visualizing the capsule 900 and/or by tracking the capsule by monitoring RF signals transmitted by the capsule 900. The remote processing device 950 may provide control signals to the control circuitry 906 in accordance with the monitoring of the capsule's 900 location for controlling one or more of the operations of the capsule 900, as described above and below.

The ultrasound probe 952 includes a transducer 954 and associated circuitry for transmitting data between the capsule 900 and the remote processing device 950 and/or another capsule. The remote processing device 950 transmits data, such as commands for remotely controlling the capsule via the probe 952. The transducer 954 and associated circuitry convert the data into vibratory signals which are transmitted to the element 510a. The element 510a and associated circuitry convert the vibratory signals into digital signals provided as data to the control circuitry 906.

Similarly, digital signals (e.g., data) from the control circuitry 906 are converted by the element 510a into vibratory signals. The vibratory signals are received by the probe 952, where the transducer 952 and associated circuitry receive and process the vibratory signals for converting them to digital signals (e.g., data) and providing the data to the remote processing device 950. The vibratory signals may further be received and processed by an element 510a in another capsule.

The medicament dispensing system 901 may include a combination of the elements 104, 106, 114, 116 and/or 130, as shown in FIGS. 1, 3, 5 and 7, and in accordance with their configuration and operation. The medicament dispensing system 901 may alternatively include a controllable MEMS medicament delivery system which is known in the art, or a MEMS medicament delivery system which is known in the art, and which is further provided with a control mechanism responsive to control signals from the control circuitry 906.

It is envisioned that the medicament dispensing system 901 may be replaced with another medical system for performing a medical function, such as a diagnostic or therapeutic medical function. Preferably the other system is controllable by the control circuitry 906.

With reference to FIG. 9A, the medicament dispensing system 901 includes at least one reservoir 960 for holding a medicament, a push or pressure mechanism 962 associated with a respective reservoir 960 for exerting a force on the reservoir 960 and/or the medicament for displacing medicament stored in the reservoir 960, and preferably at least one closure member 966, such as a MEMS microvalve or as is enabled by microfluidic systems of inkjet printers and the like. The reservoir(s) is in communication with at least one aperture 970 in the housing 102 through which the medicament can exit the capsule 900. At least one pressure sensor 968 may be provided, such as for measuring the pressure in the respective reservoir(s) 960. Respective closure members 966 may be disposed at the aperture(s) 970 for controlling flow of the medicament through the aperture(s) 970, and/or at an open end of the respective reservoir(s) 960, and/or along a conduit between a reservoir 960 and aperture 970.

The medicament delivery system 901 is controllable by the control circuitry 906, such as by controlling the respective pressure mechanisms 962 and/or the at least one closure member 966. Control of the medicament control system 901 may include controlling the timing of delivery of the medicament, the amount of medicament delivered, the rate of delivery of the medicament and/or the force at which the medicament is delivered. Preferably, the medicament delivery system 901 is controllable for facilitating controlled intermittent delivery of the medicament.

The at least one closure member 966 is preferably controllably opened or closed, wherein when open, the closure member 966 preferably allows fluid to flow in only one direction. In one embodiment, the closure member 966 includes a MEMS valve including a microvalve, such as a fluidic transistor, and an associated microvalve actuator mechanism. The microvalve is preferably in a normally closed state (e.g., the microvalve substantially does not allow flow through the microvalve in either direction) and is actuatable to an open state (e.g., the microvalve allows flow of medicament for exiting the reservoir 960 and/or the capsule 900) by the actuator mechanism for a selected duration of time for allowing the flow of fluid. Preferably the rate at which the medicament flows through the microvalve when in an open state is selectable and controllable. Control of the actuator mechanism and/or the microvalve is provided by the control circuitry 906. Examples of microvalves known in the art include microvalves designed by Redwood Microsystems™, and microvalves described at www.cornell.edu/2003cnfra/2003cnfra172.pdf.

The actuator mechanism may include a micromotor which may be powered by the power source 508 for mechanically opening and closing a moveable mechanism within the microvalve. The size of the opening is preferably selectable for controlling the rate at which the medicament flows when in an open state. Alternatively, the actuator mechanism may control displacement of the medicament with respect to an opening in the microvalve. The actuator is preferably controllable for controlling the degree of displacement and thus the rate at which the medicament flows when in an open state.

The microvalve may include structural materials, such as Si, SiO₂, SiN, Ti, and/or TiNi, and gasket materials, such as PDMS, Polymide, Polycoarbonate, Parylene and/or silicone rubber. The actuator mechanism may include, for example, electrostatic, magnetic, piezoelectric, bimetallic, shape memory alloy (SMA), pneumatic and/or thermopneumatic construction and functions.

Another exemplary closure member 966 includes a valve having at least one controllable artificial muscle made of a polymer that expands or contracts in response to an electrical signal for substantially plugging or unplugging an aperture. Similarly, the expansion and contraction of the artificial muscle may be included in the actuator mechanism for controlling displacement of the medicament for controlling flow thereof. Electrically activated artificial muscles for opening and closing a reservoir in a biological MEMS system are described in IEEE Spectrum, October 2004, pp 49-53.

The controllable valve 106 of FIGS. 1, 3, 5, 7 and closure members, (e.g., MEMS valves and microvalves) described below may be configured substantially in accordance with the description with respect to the structure and function of closure members 966. It is envisioned that the normal state (e.g., opened or closed state) for the particular closure member be selected in accordance with design choice.

In one embodiment of the disclosure, the reservoir 960 may include a deformable chamber responsive to pressure from the pressure element 962. The pressure mechanism 962 includes a displaceable and/or expandable member which exerts pressure on the reservoir 960 or medicament for displacing medicament held in the reservoir 960 in order for the medicament to exit the reservoir 960. For example, in the embodiment shown in FIGS. 1, 3, 5 and 7, the pressure mechanism includes a piston-type member 130 and a biased element, such as a spring 116, that exerts a fixed force on the piston-type member 130 for displacing the piston-type member 130 and exerts pressure on the reservoir 104, which has an open end covered by valve 106. Dispensing of the medicament may be additionally controlled by controlling the valve 106.

Preferably, the open end of the reservoir 960 is coincident with one of the apertures 970 of the housing 102, and one closure member 966 provides closure thereto. When the closure member 966 is in an open state, medicament exiting the reservoir 960 (e.g., due to pressure exerted by the pressure mechanism 962) passes directly from the reservoir 960 through the aperture 970 and into the ambient surroundings of the capsule 900. In order to be dispensed the medicament does not need to pass through any conduits or additional closure members once it exits from the reservoir 960. By configuring the open end of reservoir 960 to be coincident with the aperture 970 (e.g., for controlling the pressure mechanism 962 and/or the closure member 966), any delay from the time a control signal is generated for dispensing of the medicament until the medicament is dispensed is minimized. Otherwise delays could be caused by the medicament traversing additional conduits or closure members after exiting the reservoir, and/or by control and operation of the additional control members. Furthermore, by configuring the open end of the reservoir 960 to be coincident with the aperture 970 there is no residual medicament left in any conduits, and thus there is a benefit for precise dosing of the medicament.

In one embodiment of the disclosure, as described in U.S. Pat. No. 5,318,557, assigned to Elan Medical Technologies, Limited, the pressure mechanism 962 may include a chamber holding an electrolytic cell which generates a gas when electrical current is passed there through. As pressure within the chamber increases, pressure is exerted on the deformable reservoir 962 for forcing delivery of medication through an open end of the reservoir 962. In another embodiment of the disclosure, the pressure mechanism 962 may include an artificial muscle formed of a polymer that controllably expands or contracts in response to an applied electrical signal for applying pressure to the deformable reservoir 962 and/or the stored medicament.

In another embodiment of the disclosure, the pressure mechanism 962 may include an osmotic membrane which enlarges at a slow rate when it is exposed to a liquid. An osmotic pressure element is described in U.S. Pat. Nos. 4,519,801; 4,612,008; 4,783,337; and 5,082,668, all assigned to Alza Corporation.

With reference to FIG. 10, a capsule 1000 is shown having a controllable osmotic pressure element 1002. The osmotic pressure element 1002 exerts pressure on a deformable reservoir 1004 for dispensing medicament through aperture 1005 of the reservoir 1006 in response to absorption of fluid by the osmotic pressure element 1002. A housing 1008 of the capsule 1000 includes a first aperture 1010 having a controllable closure member 1012, such as a microvalve and associated actuator mechanism, responsive to control signals from control circuitry 906 for controllably allowing fluid to enter the housing 1008 from the environment of the capsule 1000. The size and/or frequency of opening of the closure member 1012 are controlled by the control circuitry 906. Closing closure member 1012 prevents additional fluid from entering the housing 1008 for absorption by the osmotic pressure member 1002, and thus terminates further enlargement thereof. A time lag may exist between closing closure member 1012 and terminating enlargement of osmotic pressure member 1002, which may be compensated for by the control circuitry 906.

By opening the closure member 1012, enlargement of the osmotic pressure member 1002 may be resumed for intermittent dispensing of the medicament through aperture 1005. A time lag may exist between opening closure member 1012 and resuming enlargement of the osmotic pressure member 1002, which may be compensated for by the control circuitry 906.

The housing 1008 is further provided with a second aperture 1014 in fluid communication with aperture 1005, wherein medicament dispensed from aperture 1005 passes to aperture 1014 through which it is dispensed to the environment of the capsule 1000. The pressure exerted on the reservoir 1004 for dispensing medicament therefrom is related to and responsive to the amount of fluid entering housing 1002 from the environment of the capsule 1000, which is controlled by the controlled operation of the closure member 1012. The apertures 1014 and 1005 may further be provided with controllable closure members 1016, similar to closure member 1012, which are responsive to control signals from the control circuitry 906 for further controlling of dispensing of the medicament to the environment of the capsule 1000.

Control circuitry 906 and other circuitry, such as a communication assembly, a power source, etc., may be provided within a sealed compartment 1018 which prevents fluid from entering and interfering with the enclosed circuitry. Communication between control circuitry 906 and closure members 1012 and 1016 may be via wireless communication and/or via wired communication, where the wires and connections are resistant to fluids.

With reference to FIG. 9B, the capsule 900′ includes medicament dispensing system 901′ which includes at least one micropump 972 and/or microvalve and associated actuator mechanism 974 in fluid communication with an aperture in the housing 102 of the capsule for controlling dispensing of medicament from the capsule. It is envisioned that the micropump 972 and/or the microvalve 974 may include, incorporated respectively therein, a reservoir, a pressure mechanism and/or a valve. With respect to the microvalve 974, the actuator mechanism may provides at least a portion of the displacement action, such as provided by the pressure mechanism 962 of FIG. 9A. The micropump 972 includes, for example, a micro-peristaltic pump. In an exemplary micro-peristaltic pump known in the art, at least one heater suspended in a thermopneumatic is disposed in a combination of stacked silicon wafers (e.g., a channel wafer, a membrane wafer and a heater wafer). Heating of the fluid causes deflection of a membrane which controls flow of the medicament. The heating of the fluid is provided, for example by applying a controlled voltage, where control is provided by the control circuitry 906.

In one example the micropump includes a thermodynamic pump similar to pumps used for heat-driven inkjet printers. For a small capsule 900′ having a small power source, such as an ingestible capsule, power consumption may limit duration of active use of the thermodynamic pump. In a larger capsule having a larger power source, such as an implantable capsule, the power consumption is less of a limitation. Furthermore, thermal damage to medicament may be minimized, such as by providing insulation or a cooling system. For example, the device(s) for generating heat having an expanding/contracting fluid for causing an expansion and pumping action may be provided in a closed system (similar to an air-conditioning system) which is separated by a membrane, which preferably includes an insulator, from the storage and passage ways for the medicament.

With reference to FIG. 11, a closure member assembly 980 is shown including two or more closure members 964 disposed about the capsule 900. The respective closure members 966 provide selectable closure to respective associated apertures 970 disposed at various positions of housing 102, such as for selectably dispensing at least one medicament from the capsule in different directions. The closure members 964, similar to closure member 966, are shown to be in fluid communication with one reservoir 960 by way of a channel 982 (which may have several branches) for dispensing one medicament. It is envisioned that respective closure members 964 may be in fluid communication with different reservoirs for delivering more than one medicament. The closure members 964 are preferably addressable and independently controlled by control circuitry 906 for dispensing the medicament (or a selected medicament) in a selected direction via one or more closure members 964. In some applications it is preferable for the opening of the reservoir 960 to be as close as possible to the aperture 970 within the housing 102, or for the channel 982 to be as short as possible for minimizing delays in dispensing the medication out of the capsule 900. A controllable closure member 984, similar to closure member 966, may be provided for controlling flow of medicament through the open end of the reservoir 960 into the channel 982.

Furthermore, the closure members 964 and/or the apertures 970 may be disposed about the capsule 900 so that when dispensing the medicament through a plurality of the closure members 964 a ring or other pattern is formed of deposited medicament on the anatomy of the patient. The force with which the medicament is dispensed may be controlled, such as by controlling pressure with which the medicament is forced through the closure members 964 and/or controlling the size of the opening of the respective closure members. The closure member assembly 980 may be disposed at a variety of positions about the capsule 900, such as at a tapered end or about the mid-area where the capsule 900 is wider or widest.

FIGS. 12 and 13 show a capsule 1200 and a capsule 1300, respectively, having multiple reservoirs. The capsules 1200 and 1300 are free standing capsules which are not attached structurally to a device located external to the patient. In each of the capsules 1200 and 1300 individual reservoirs are provided in respective modules which are interlocking and/or connectable electronically and/or mechanically. The respective modules may include other components of the medicament dispensing system 901 and/or circuitry, such as a communication assembly, control circuitry 906 and/or a power source. The respective modules may be prepared independently, including filling the reservoirs 960 with a medicament and/or programming the control circuitry 906, even at different locations, such as at the locations of different pharmaceutical entities. Once prepared, the respective modules may be assembled into one capsule. It is envisioned that the capsule may be prepared with the respective reservoirs, which may be filled while assembled in the capsule, such as by plugging them into one another or a base, and encasing them in a housing 102 and preparing apertures 970 in appropriate places. It is further envisioned that the reservoirs may be prepared and filled in different locations, after which the reservoirs may be placed or plugged into an already assembled or partially assembled capsule. It is further envisioned that the control circuitry 906 may be programmed prior to, during or after assembling of the capsule 1200, 1300.

First and second modules 1202 and 1204 of capsule 1200 are shown, where each module includes sufficient components for operating as a stand-alone module. Modules 1302, 1304, 1306 and 1308 of capsule 1300 are shown, where each module includes at least a portion of a respective medicament dispensing system 901. The capsule 1300 further includes space 1308 in which shared components or resources are provided. The shared components may include any combination of the antenna 502, the communication assembly 504, the control circuitry 906, the element 510a and the power source 908. Mechanical and/or electrical connectors 1310 are provided between the modules and/or the shared components, preferably for facilitating sharing of the functionality of the shared components. The electrical connectors 1310 may be configured in a variety of configurations, such in a bus configuration, a distributed configuration or a centralized configuration. The modules 1302, 1304, 1306, 1308 may all share the same components as one another, or may share different components from one another. Each module 1302, 1304, 1306 and 1308 is preferably independently controlled. For example, the modules 1302, 1304, 1306 and 1308 may be individually addressable by shared control circuitry 906.

Modules within a capsule may communicate with one other, such as via low power communication, where power used may be low relative to power used for communication between a capsule and a device located outside the body of the patient. For example, modules 1202 and 1204 may communicate with one another, and modules 1302, 1304, 1306 and/or 1308 may communicate with one another. Intra-capsular communication may be provided, for example, via wireless communication, e.g., RF or ultrasound communication, and/or via wired communication using connectors (e.g., each module having conductive contacts which couple with corresponding respective contacts of another module).

The reservoir 960 of modules 1202 and 1204, and/or the reservoirs of modules 1302, 1304, 1306 and 1308 may be provided with a sealable access 1220 through which to fill the reservoir 960 with a medicament. After filling the reservoir 960 with the desired amount of medicament the access 1220 is sealed. The access 1220 may be configured as a valve or membrane through which a syringe may deliver medicament but is resilient for closing the puncture site, forming a seal, as known in the art. The access 1220 may be provided at any location of the housing of the reservoir 960. The reservoir 960 may be sealed using a variety methods that are known in the art, such as for filling a syringe, vial, etc.

With reference again to FIG. 9A, preferably, the at least one software module 980 includes a dispenser control software module for controlling release of the medicament, in accordance with at least one predetermined condition, such as a sensed value (e.g., when a threshold value is exceeded) or a time related condition, such as at periodic time intervals. For example, the dispenser control software module controls the respective closure members 964 and 966 and/or the pressure system 962 for dispensing the medicament at regular time intervals, such as where the medicament is a contrast agent or an imaging or medical marker substance for placing markers or contrast agent depositions as fiducial marks, e.g., reference marks, along the alimentary tract.

The contrast agent may be an agent which is visible after deposition in the patient, such as via the eye, microscope, camera (such as a camera disposed in a capsule), a medical imaging modality, etc. For example the contrast agent may be barium which is visible via X-ray or CT imaging, or a paramagnetic agent which is visible via MRI imaging. The medical marker substance may be a substance, such as a carbon based ink (e.g., India ink) or methylene blue, which may temporarily or permanently stain the tissue to which it is applied as a marker.

Finding the location of an area previously identified in a diagnostic procedure, such as a diagnostic procedure performed by a capsule, for example a camera capsule combination (e.g., a camera aboard a capsule), is complicated by factors such as mobility of the small intestine. For example, it is not sufficient to describe the location of the identified area by 3D coordinates for the purpose of finding the location in a subsequent non-invasive procedure. One way to describe the location of the identified area is by specifying the time elapsed from entry of the camera capsule combination into the alimentary tract (e.g., from time of ingestion). Furthermore, it is possible to somewhat more accurately describe the location of the identified area by specifying time elapsed after traversal by the camera of a visible landmark. For example, the camera aboard a capsule may collect and optionally transmit images, so that a reviewing practitioner (e.g., a radiologist or gastroenterologist) or a computer-aided detection system, e.g., performing image matching algorithms, can detect changes in texture of the tract being traversed. Changes in texture may be correlated with entry of the camera capsule combination into different sections of the alimentary tract, such as the esophagus, stomach, duodenum (junction between stomach and small intestine), cecum (junction of small and large intestine), and rectum.

Additionally, the proportion of time elapsed between traversal of major visual landmarks can be used to further describe the location of the identified area. However, the elapsed time can be several hours through the small intestine, with variable rates of peristalsis in different sections of the small intestine, even in the same patient. This makes the described location an even less accurate estimation, such as for use in a subsequent intervention. When the subsequent intervention is an open surgery, the surgeon can often identify a visible problem by inspection, which may be time consuming, particularly for less visually obvious problems. Furthermore, not all problems are identifiable visibly. In a minimally invasive procedure, such as through the use of an endoscope or subsequent capsule (e.g., for deposition of medicament at a desired location), locating the identified area typically requires depending heavily on the described location of the area.

Using detectable marks deposited by the capsule 900 at regularly timed intervals, the location of a target area identified during a diagnostic procedure may be more accurately described prior to performing the diagnostic procedure or after performing the diagnostic procedure by describing the location relative to the deposited marks. The marks may then be used to find the location during a subsequent procedure. Use of the marks during an open surgical procedure or a minimally invasive procedure increases speed and accuracy in locating the area. In a minimally invasive surgery the marks may function analogously to ‘mile markers’ on a highway for finding the location of the area to be treated. When the minimally invasive procedure includes dispensing medicament from an electronically controlled capsule, dispensing of the medicament may be triggered by counting marks as they are passed.

In one example, a camera capsule combination is ingested for traversing the alimentary tract. A capsule 900 for dispensing a series of marks at regular intervals is ingested after a known time interval “s”, such as ten minutes or more. This way the capsule 900 follows the camera capsule combination through the alimentary tract without interfering with or catching up with the camera capsule combination. There is a high degree of variability during traversal of the alimentary tract for the time to transit through the stomach. Therefore, a reference location is used at which timing is begun (time=0) for both the camera capsule combination and the capsule 900. Preferably, the reference location is traversed after exiting the stomach, such as upon entering the small intestine (e.g., at the duodenum, which is about 25 cm in length for an adult). For example, entry into the duodenum may be determined by the cameral capsule combination by identifying changes in texture shown in acquired images, and by the capsule 900 based on pH readings sensed by a pH sensor aboard the capsule 900. It is contemplated that the camera capsule may include a pH sensor as well, and may detect the reference location using output from the pH sensor.

In operation, when the camera capsule reaches the reference location the timing is synchronized and timing begins with time=0. Synchronization and/or timing can be performed by intercapsular communication between capsule 900 and the camera capsule and/or a remote processing device. The time at which the capsule 900 reaches the reference location is called “s”.

Images acquired by the camera capsule combination are analyzed by a remote processor, such as remote processing device 950, even after the camera capsule combination is expelled from the patient. The area to be targeted for a subsequent procedure may be determined based on the acquired diagnostic images. The time “t” that it took for the camera capsule combination to travel from the reference location and reach the target area is determined. The time that the capsule 900 passed the target area is determined as “t”+“s”. The location of the target area relative to a respective specific mark of the series of marks is determined for use during a subsequent procedure. For example, a subsequently ingested capsule (e.g., a third capsule) for performing the subsequent procedure can count marks for locating the specific mark and release a medicament at the location of the target area which is known relative to the specific mark. Accordingly, the medicament, such as an anti-inflammatory medicament, may be applied directly to the target area (which may be an inflamed area, for example) without applying the medicament to healthy tissue unnecessarily. This method may be used for locating several target areas during the subsequent procedure.

As described above, the marks may be visible during open, endoscopic or laparoscopic surgery, visible during imaging, sensed by a subsequent capsule capable of sensing marks or detected during imaging for tracking subsequently ingested capsule. Sensing or detection of a mark or a predetermined number of marks during traversal of by the subsequent capsule may trigger enablement or activation of one or more functions by the subsequent capsule. The subsequent capsule may be configured for performing a diagnostic procedure or therapy based on detection or sensing of the marks. When the marks are generated as fiducial markings, the subsequent capsule may sense the marks or an imaging procedure may, and perform a diagnostic procedure. Diagnostic information may be correlated with the marks and their positions, or a therapy may be provided at regular intervals in accordance with sensing or detection of the marks.

Furthermore, the markers and/or contrast depositions may be sensed (e.g., by imaging or by a subsequent capsule) for deriving information about the peristaltic action of the alimentary tract or a portion thereof, which may include studying the spatial intervals between the markers or contrast depositions and correlating the spatial intervals with the temporal intervals at which the markers or contrast depositions were dispensed from the capsule 900.

Different contrast agents may be controllably dispensed from different capsules or from different reservoirs within capsule 900. Contrast agents having different colors may be dispensed, for example, for distinguishing between subsequent depositions and/or for visualizing twists and turns of areas of the alimentary tract, such as the colon. Likewise, contrast agents used for different modalities may be dispensed. The amount, location or timing of dispensing of a contrast agent or marker may be controlled, for example, for dispensing the contrast agent or marker in a region that is suspicious pathologically, such as was viewed in an image marked by a mark left by a previous capsule, or was sensed by a sensor.

Multimodal registration for 3D images is known. Registration using a fourth dimension of time is known for a single imaging modality, such where first and second 3D images are acquired with a time interval in between the acquisitions, and registration is performed between the first and second images. In the present disclosure, mark depositions deposited at regular time intervals may be used for registration between images generated by even two or more imaging modalities and/or for registration of images acquired at different points in time, thus achieving multimodal registration in a fourth dimension. Accordingly, registration over spatial as well as temporal planes and multimodalities can be achieved. Registration over multimodalities and the fourth dimension can improve accuracy of co-registration and provide additional information relative to the use of one imaging modality.

As described above, the capsule 900 may be an implantation device or an ingestible device. The implantation device may be placed in a desired location for controlled intermittent or prolonged dispensing of the medicament, sensing physical properties, and/or communicating with the remote processing device 950 and/or another capsule 900 internal to the patient. The implantation device may be placed in various parts of the body, e.g., brain, liver, breast, etc., percutaneously or intramuscularly, such as via a catheter placed through a percutaneous tissue tract. The implantation device may controllably dispense a medicament, such as a pharmaceutical, e.g., antibiotics or hormones, which requires or is best administered percutaneously over an extended time period (e.g., for a week or more). Exemplary applications for the implantation device include administration of growth hormone, insulin, birth control, etc. The medicament dispensing system 901 may be controlled by the control circuitry 906 and/or by the remote processing circuitry 950 in accordance with sensed properties, patient feedback, a pre-programmed schedule, etc.

In another application, the implantation device is placed surgically (e.g., open, endoscopically or laporoscopically) close to a target (e.g., a tumor) such as for controlled dispensing of a medicament directed at the target, such as for pre-surgical or post-surgical treatment, or in lieu of treatment. Since the implantation device may be as small as an ingestible device, the surgical implantation procedure may be simplified.

Implantation of the capsule 900 may be especially useful for the long-term release of chemotherapeutic agents. Recent research indicates that some tumors require 2-3 days to uptake the amount of chemotherapeutic agent required to kill cancer cells. The relatively long uptake time may be due to the chaotic way that tumors create neo-vascularization which produces an inefficient uptake and release of blood (also known as “wash-in/wash-out”). Diagnostic imaging systems in conjunction with contrast agents make use of the comparative uptake inefficiency for highlighting suspected lesions which retain the contrast longer. However, due to the effects of the chemotherapeutic agents on healthy tissue a patient typically cannot stand more than a few hours of application of the chemotherapeutic agent. If a cancer is localized, such as in a single tumor or lesion, an electronically controlled capsule may provide gradual controlled release of the chemotherapeutic agent over a long period of time as required for uptake by the tumor. Furthermore, the chemotherapeutic agent may be directed at the tumor for minimizing unwanted uptake of the chemotherapeutic agent by healthy tissue.

With respect to FIG. 9C, capsule 900 configured as an implantation device is provided with a customized nozzle 982 connected to the medicament dispensing system 901. The nozzle 982 is shaped and sized to correspond to the shape and size of the lesion shown at 980 for directing the chemotherapeutic agent toward the legion, and minimizing application of the chemotherapeutic agent to health tissue. The nozzle 982 operates similarly to a, porous watering can nozzle, by directing medicament, e.g., a chemotherapeutic agent. dispensed by the medicament dispensing system 901 toward the lesion. The open end of reservoir 960 is in fluid communication with the nozzle 982, such as via a conduit 984. The nozzle 982 is provided with a plurality of apertures or pores 986. As medicament is dispensed from the reservoir 960, at least a portion of the medicament is directed through conduit 984 into the nozzle 982 and dispensed through apertures 986 for dispensing the medicament directly onto the lesion along the surface of the lesion. The nozzle 982 may be shaped and sized prior to implantation using information about the shape and size of the lesion 1980, such as obtained from acquired images. Furthermore, the nozzle may be formed of a pliable material that can be shaped during the implantation procedure. The nozzle may be shaped, for example, to surround the lesion for dispensing medicament onto a maximum amount of the surface area of the lesion 1982 and minimizing contact of the medicament with untargeted or healthy tissue.

Where the capsule 900 is ingestible, the capsule 900 is moved along the alimentary tract where it may perform diagnostic or therapeutic procedures, and has access to areas reachable by an endoscope as well as areas that are difficult to reach using an endoscope. Just as importantly, the capsule 900 is less invasive than an endoscopic procedure, and further does not require sedation of the patient or a hospital stay, etc.

With reference to FIG. 9A, sensors 904 of sensor module 902 may be disposed on the shell 102 and/or may be enclosed within the shell 102, where a controllable closure member provides exposure of the sensor 102 to the environment of the capsule 900. Accordingly, the sensors 904 may be permanently exposed to the environment of the capsule 900, or may be controllably exposed. The sensors 904 generate sensing signals corresponding to the sensing. The sensing signals are sent to the control circuitry 906 and/or the remote processing circuitry 950. Operation of the sensors 904 may be controllably enabled, such as for avoiding generating or processing data that is not of interest, or just sampling data of interest, for conserving resources, such as processing and/or input/output (I/O) resources. It is contemplated that the capsule 900 may be intended for diagnostic purposes only and does not include the medicament dispensing system 901.

One method of controlling operation of the sensors 904 includes providing individual sensors 904 or groups of sensors 904 with a controllable and closeable enclosure. For example, the sensor(s) 904 may be disposed within a chamber having a controllable MEMS closure element, such as a hatch or a valve, which may be controlled to selectively expose the sensor to the environment of the capsule 900. The control circuitry 906 may generate control signals for controlling the closure element, where the control signals may be generated, for example, in accordance with at least one predetermined condition, such as receipt of instructions received from the remote processing circuitry 950, a sensed condition sensed by exposed sensors 904 (e.g., when a threshold value is exceeded), a timing schedule, etc. When a sensor 904 is not exposed to the environment of the capsule 900, the signals generated by the sensor 904 may not be used, thus disabling the sensor 904. Alternatively, signals generated by a sensor 904 that is not exposed may be used for a special purpose, such as for a control or reference value.

Another method of controlling operation of the sensors 904 includes selectively enabling propagation of the sensing, which may be implemented using at least one analog or digital device, such as a switch, along the propagation path of the sensing signal. In another method of controlling operation of the sensors 904, a respective sensor 904 may be disabled, such as by obstructing power delivery to a sensor 904 that requires power for operating and/or transmitting signals. In still another method of controlling operation of the sensors 904, the processing of the sensing signals may be selectively enabled.

The sensing signals and sensor enablement data describing control of operation of the sensors 904 may be stored by the capsule 900 and retrieved from the capsule once expelled from the patient and/or transmitted to the remote processing circuitry 950 for analysis. Analysis may include correlation with time, which may further include correlation with distance traveled by the capsule 900 through the alimentary tract. Accordingly, the data generated by the sensors 904 may be used for generating a mapping of sensed information versus time, or a spatial mapping of sensed information versus location of the capsule 900 along the alimentary tract.

In one embodiment of the disclosure, one of the sensors 904 is a pH sensor for sensing pH levels, for example as the capsule is moved along the alimentary tract, and one of the software modules is a pH control software module. The pH control software module monitors sensing signals output by the pH sensor for determining when the capsule 900 has reached a desired location in the alimentary tract, upon which a control signal is transmitted for controlling a function of the capsule 900. The control signal may be provided, for example, to the medicament dispensing system 901 for dispensing the medicament or a portion thereof. The pH control software module may continue to monitor the pH levels and dispense the medicament in response to the pH levels for delivery of the medicament at a desired rate and at desired locations along the alimentary tract in accordance with the determined pH levels.

The pH readings by the pH sensor advantageously trigger dispensing of the medicament, where advantages include the ability to transport the medicament payload of the capsule 900 to a desirable position, which may be past the stomach where absorption to the blood stream of some medicaments is poor and proteins are destroyed. Thus, dispensing of the medicament may be delayed until the capsule 900 reaches a desired position where absorption is maximized, such as the duodenum or far along the small intestine and/or in the large intestine. It may be desirable to control traversal of the alimentary tract by the capsule 900 (e.g., preferably after eating and not before, as ingested food would interfere with positioning of the capsule 900), as described further below with respect to FIG. 16, for keeping the capsule in the desired position during which the medicament is dispensed. For example, the duodenum, which is relatively short (approximately 25 cm) has a high surface area due to villi, and is highly vascular. Many current medications and vitamins. are absorbed primarily in the duodenum.

The actual pH level, changes in the pH level and/or rate of change of the pH level may be monitored for determining the location of the capsule 900 and for controlling dispensing of the medicament. The pH level of the stomach is typically about 2.0, ranging from 1-3 in normal healthy humans. The pH level of the small intestines is about 6. The pH level of the duodenum typically 6-6.5 pH, but can reach 7 or 8. The pH level of the next two parts of the small intestine, the jejunum and ileum, gradually rise in pH to 7.5 The pH levels of the large intestine drops to 5.5-7. Processing of control signals for controlling dispensing of the medicament may be performed by the control circuitry 906 or a remote processor, such as the remote processing device 950. The processing of the control signals may include consulting a mapping (e.g., a look up table, a continuous mapping, a searchable database, etc.) of positions along the alimentary tract versus pH levels (or ranges thereof), and using the mapping to determine the location of the capsule 900 in accordance with the current pH level, change in pH level or rate of change of pH level.

In the small intestine vascularity is ninety percent, which is substantially directly provided to the liver, where medication is metabolized and thus removed from the bloodstream. Medicament delivered in the large intestine is highly bioavailable and less toxic to the liver, since in the large intestine ninety percent of the circulation flows through the circulatory system first, and later to the liver.

It is envisioned that more than one capsule 900 may be used for dispensing medicament(s) to the patient, where it is important for one of capsules 900 to know the status of the other capsule 900. For example, consecutively ingested capsules 900 or multiple implantation devices may provide a continuous dose or combined dose of a one or more medicaments, where it is critical that delivery of the dose be coordinated, such as provided one at a time, without overlapping, to avoid overdosing. Accordingly, it is advantageous for one capsule 900 (e.g., a second capsule) to be aware if a capsule 900 previously administering a dose (e.g., a first capsule) has stopped dispensing a medicament, such as due to a depleted reservoir, a depleted battery or having exited the patient's alimentary tract.

It is further envisioned that the first capsule emit a signal (a continual signal or discrete signals) when dispensing the medicament, where the signal is detectable by the second capsule. When the second capsule detects that the first capsule is no longer emitting the signal (e.g., such as due to having exited the alimentary tract or depleted its payload), the second capsule commences to dispense the medicament. Alternatively, the first capsule may recognize, detect or sense that it is about to terminate or has terminated dispensing medicament, and thereupon emits a signal indicating that the second capsule should take over by dispensing its medicament. Alternatively, the first and second capsule may be programmed to dispense medication in consecutive dispensing cycles, where the first capsule stops dispensing and the second capsule commences dispensing when a predetermined condition is met, such as the passing of a predetermined time interval (e.g., which may be determined on an absolute or relative basis) or sensing of a property.

In another embodiment of the disclosure, with reference to FIG. 14, a capsule 1400 is provided having at least one chamber 1402 in which to store an ambient substance, typically bodily fluids. The capsule 1400 is a free standing capsule which is not attached structurally to a device located external to the patient. Preferably, the chambers 1402 are vacuum filled or provided with a negative pressure. Each chamber 1402 has an aperture that is in fluid communication with an aperture 970 in the housing 102, where at least one of the aperture in the chamber 1402 and the aperture in the housing 102 is provided with an associated closure member 1406 which is controlled by the control circuitry 906. Closure member 1406 may be similar, structurally and operationally, to closure member 966 of FIG. 9. Preferably the aperture of the chamber 1402 is coincident with the aperture 970 of the housing 102 and one closure member 1406 provides closure to thereto. The software module 980 includes a sampling software module for controlling the closure member 1406.

By providing the aperture of a respective chamber 1402 to be coincident with the aperture 970 of the housing 102 with one closure member 1406 providing closure thereto, ambient fluid entering the chamber 1402 passes directly into the chamber 1402 when the closure member 1406 is in an open state. Accordingly, the ambient fluids entering the capsule 1400 do not have to pass through additional conduits or closure members, minimizing any delay from the time a control signal is generated to open the closure member 1406 until a sample is acquired. Furthermore, residual loss of any of the acquired sample which could occur when traversing any additional conduits is minimized.

The exemplary capsule 1400 is shown to have dividers 1408 for defining seven collection chambers 1402. Furthermore, the dividers define an additional area 1404 in which components of the capsule 1400 are disposed, including, for example, the control circuitry 906, the communication assembly 504, element 510a and power source 908. The chambers 1402 are preferably fluid resistant for not allowing entry of fluid other than through the respective closure member 1406. Respective chambers 1402 may be provided with a reagent for beneficially reacting with fluids that enter the chamber 1402, where the reagent may be deposited within the chamber 1402 or provided as a coating along an inner wall of the chamber 1402.

The dividers are formed of a non-permeable material which separates the respective chambers 1402 from each other or other areas of the capsule without allowing fluid communication therebetween so that each chamber 1402 is impervious to fluid. It is envisioned that the collection chambers 1402 and/or dividers 1408 may have different configurations than shown. For example, the housing 102 may provide an interior or exterior wall for the chamber, and the capsule 1400 may further house a combination of other components, such as a medicament dispensing system, sensors or a camera. Additionally, an area for housing the other components of the capsule 1400 may be provided in-between one or more chambers 1402, or in a defined center area of the capsule 1400.

The closure member 1406 is closed for blocking entry of a substance until it is desired to acquire a sample, such as upon fulfillment of a predetermined condition, and for maintaining an acquired ambient substance within the chamber. After the capsule 1400 exits the alimentary tract of the patient, the capsule 1400 is retrieved and the contents retained within the chambers 1402 are analyzed. Accordingly, the contents of the chambers 1402 acquired by sampling bodily fluids along the alimentary tract of the patient may be analyzed in a full scale laboratory.

The closure member 1406 may further include a hatch which may be opened or closed for allowing or preventing, respectively, the flow of fluids into the chamber. A micromotor controllable by the control circuitry 906 is provided for actuating the hatch. Other technologies are also envisioned to open and close hatches, such as the use of small electronic ‘muscles’ that open or close hatches of small chambers, or function as the hatch itself.

A large chamber may benefit from having at least two apertures in fluid communication with (and preferably coincident with) respective apertures 970 in the housing 102 and provided with closure via respective closure members 1406, particularly for relatively highly viscous ambient substances. The multiple closure members 1406 of a chamber 1402 may be positioned at opposite ends of the associated chamber 1406.

In a simplified embodiment, the respective closure members 1406 are in a normally open state. The sampling software module controls the closure member 1406 to close upon fulfillment of at least one predetermined condition, such as a time-related condition, a sensed condition, or receipt of a command, such as from another processor located in a different capsule or in a device outside the patient's body. For example, the sampling software module may control the closure member 1406 to close upon sensing that the capsule 1400 is about to exit from a particular location. In a preferred embodiment, the sampling software module controls the respective closure members 1406 independently to open and close.

In another exemplary embodiment, the closure member 1406 associated with a selected chamber 1402 may be independently controlled to open and close for capturing a sample at the location where the capsule 1400 is currently situated along the alimentary tract. Closure members 1406 associated with respective chambers 1402 may be independently controlled for opening and closing one at a time in a sequential manner (e.g., in a pattern, such as a spiral) for capturing samples at different intervals, and accordingly at different positions along the alimentary tract. Preferably a negative pressure is provided within the chambers 1402 for assisting fluid to enter the chamber when the associated closure member 1406 is opened. Opening of the closure member 1406 may be very brief and very small in size. The intervals may be timed intervals, e.g., regular intervals, and/or may be determined in accordance with at least one condition, such as a sensed condition and/or the tracked location of the capsule 1400 by an external device.

Preferably, the sampling software module controls opening and closing of the individual closure members 1406, the size of the opening of the individual closure members 1406 and/or the duration of opening the individual closure members 1406 in accordance with at least one condition such as a timed condition, sensed condition, received instructions from a remote device, etc. Depending upon the requirements of the analysis to be performed on the samples acquired, the amount of ambient substance required per chamber 1402 may vary. The individual chambers 1402 may be equipped with a sensor for sensing the presence of a fluid sample or a volume of the sample, which may function to trigger the associated closure member(s) 1406 to close. The sampling software module may be programmed for actuating the individual closure members 1406 in accordance with the sample size requirements, the patient's anatomy, etc.

It is envisioned that the capsule 1400 be further provided with a pressure mechanism for establishing a negative pressure in the respective chambers 1402. Preferably the pressure mechanism is controllable for establishing a selected, controlled pressure. Furthermore, preferably the pressure mechanism is controllable for independently controlling the pressure of the respective chambers 1402.

It may be preferable that the capsule 1400 be oriented so that the closure members 1406 of the respective chambers 1402 are directed opposite the flow of bodily fluids through the alimentary tract so that the fluid flows towards the closure members 1406 and is directed into the chamber 1402 when an associated closure member 1406 is opened. The capsule 1400 may be provided with a weight assembly 1430 disposed at one of the tapered ends of the capsule 1400 for biasing the weighted end to be directed downward in the direction of the flow of fluid through the alimentary tract. The capsule 1400 may be provided with a marking 1432 on the outside of the capsule 1400 for enabling proper orientation of the capsule 1432 when ingesting or opening the capsule 1400 (such as in a laboratory setting) and/or for indicating which chamber 1402 holds the first sample acquired. The closure members 1406 may be controllable, such as by the control circuitry 906 in response to control signals from a separate device for opening in a sequential manner that corresponds to the sequence in which the samples were acquired for providing access and removal of the samples in the proper order for analysis thereof.

Advantageously, the capsule 1400 is capable of sampling different areas of the alimentary tract. During analysis, if a suspicious substance, such as blood, is detected in one of the samples acquired, it is possible to determine the time and location that the sample was acquired (e.g., from the time and/or location of the capsule when the closure member(s) 1402 associated with the chamber 1402 storing the sample was opened and/or closed). For example, determination of the capsule's location at the time of the sampling may be in accordance with the interval of time passed between ingestion of the capsule and opening/closing of the opened closure member 1406, statistical baseline information for similar patients, a triangulated location using signals emitted by the capsule 1400 (e.g., RF signals), and/or images (X-Ray, MRI, etc.) acquired during the capsule's journey through the alimentary tract.

The capsule 1400 may include an alert device 1440, the software module 980 may include a retrieval alert software module, and one of the sensors 904 may be an expulsion sensor which is capable of sensing when the capsule 1400 is expelled or close to being expelled from the body of the patient. The retrieval alert software module receives sensing signals from the expulsion sensor and determines when the sensing signals are indicative that the capsule 1400 is expelled or close to being expelled. Thereupon, the retrieval alert software module generates a control signal which is provided to the alert device 1440 for activation thereof.

The expulsion sensor may be a sensor for sensing a change in the environment of the capsule 1400, including a change in the environment of a stool in which the capsule 1400 is situated during expulsion, e.g., during entry into the anal canal or expulsion therefrom. The sensor may sense, for example, a change in pressure, a change in lighting conditions, and/or a change in temperature.

The alert device 1440 may be a MEMS vibrator for providing a sensory alert to the patient; an audio device for emitting a recognizable sound; and/or medicament which is released in conjunction with the medicament release system 901 as shown in FIG. 9A for release after expulsion, where the medicament is a substance that will alert the patient, such as a concentrated dye, preferably fluorescent, or a concentrated substance having a strong distinguishable odor. The alert is beneficial for alerting the patient or caretakers thereof that the capsule 1400 was safely expelled, and or for retrieving the capsule when desired. The alert device 1440, expulsion sensor and the retrieval alert software module may be included with a variety of capsules, such as a capsule having a camera on board, etc.

With reference to FIG. 15, a capsule 1500 is shown which is capable of sensing marks, such as marks left by a previous capsule. The capsule 1500 is a free standing capsule which is not attached structurally to a device located external to the patient. The capsule 1500 is included with a mark detection system 1502 which includes a light source assembly 1504 and a photo detector assembly 1506. The mark detection system 1502 uses MEMS circuitry equivalent to circuitry found in optical code detectors, such as laser-based optical code readers or imaging-based optical code readers. Since the objective of the mark detection system 1502 is to differentiate between unstained tissue and tissue stained by a blob of ink, a high degree of precision or decoding processing is not required in the mark detection system 1502 or for the processing of signal generated thereby. The light source assembly 1504 includes at least one light source, such as a light emitting diode (LED), a xenon tube or a laser source. The photo detector assembly 1506 includes at least one photo detector for sensing incident light and generating a corresponding sensing signal, which preferably includes a minimal number of photo detectors, such as one or two rows of photo detectors or one photo detector. The photo detector assembly 1506 may further include associated circuitry for outputting a digital signal that corresponds to the sensing signal. A window 1510 is provided in the housing 102 for facilitating transmission of light through the housing 102 from the light source 1504 or to the photo detector assembly 1506.

In operation, the light source assembly 1504 emits at least one light or laser beam which impacts and is reflected from a wall of the alimentary tract near the capsule. The wall will have different light reflectivity properties, depending if it is stained with a mark or is unstained. The light source assembly 1504 may further include a scanning assembly for deflecting the beam for scanning the beam across an arc. Orientation of the capsule, such as via weighting or steering as discussed elsewhere in the present disclosure, may be desired for aiming the light source or positioning the photo detectors in a desired position. Since the mark may be formed as a ring around the alimentary tract, aiming of the light source and/or deflecting of the light beam may not be necessary.

The photo detector assembly 1506 detects reflected light incident on the photo detectors of the photo detector assembly 1506 and generates a corresponding light sensing signal. The associated circuitry processes the corresponding light sensing signal, such as for buffering, amplifying, filtering and/or converting from analog to digital and outputs a digital signal that corresponds to the light sensing signal. The capsule 1500 further includes at least control circuitry 906 and preferably antenna 502, communication circuitry 504 and/or transducer element 510a for facilitating communication between the capsule 1500 and a processing device remote from the capsule 1500, such as another capsule or the remote processing device 950. The control circuitry 906 analyzes the digital signal output by the photo detector assembly 1506 or transmits the digital signal to the remote processor for determining reflectivity properties of the surface which reflected the sensed light. The light reflectivity properties of the target from which the light beam is reflected (e.g., deposited medical mark on tissue or unmarked tissue of the alimentary tract) affect the waveshape of the corresponding sensing signals. Accordingly, the light reflectivity properties can be determined in accordance with the waveshape of the analog or digital form of the sensing signal.

The associated circuitry or a portion thereof may be provided with the remote processing device 950 for performing any additional processing necessary on the sensing signal output by the photo detector assembly 1506. The control circuitry 906 and/or the remote processing device 950 process the sensing signal generated by the photo detector assembly 1506 for determining reflectivity properties associated with the incident light. The processing of the sensing signal may be performed by analog or digital circuitry, and is preferably performed by digital circuitry processing the digital signal that corresponds to the sensing signal.

Processing of the sensing signal preferably includes generating a first control signal when the determined reflectivity properties indicate that the incident light was reflected from a medical mark deposited by a preceding capsule. A second control signal is generated when the determined reflectivity properties indicate that the incident light was reflected from tissue of the alimentary tract unmarked by a deposited medical mark. Accordingly, control of a device, function, or activity may be provided in accordance with sensing by the capsule 1500 of deposited medical marks which were deposited by a previous capsule.

With reference to FIGS. 16-18, another embodiment of the disclosure is shown. Ingestible capsule 1600 is provided with a braking system 1601 including at least one gas pressurization module 1602 and at least one balloon 1604, where inflation of the at least one balloon 1604 during traversal of the alimentary tract controls traversal of the capsule 1600, e.g., slows or stops movement of the capsule 1600 through the alimentary tract. Additionally, inflation by a selectable amount of selected balloon(s) 1604 of the at least one balloon 1604 may assist in steering and/or positioning the capsule 1600, such as for orienting the capsule 1600 in a desired orientation. The capsule 1600 is a free standing capsule which is not attached structurally to a device located external to the patient.

The use and construction of balloon and catheter combinations (e.g., balloon catheters) is well known in the medical art, as described for example in U.S. Pat. No. Re. 32,983 issued to Levy and U.S. Pat. No. 4,820,349 issued to Saab. Balloon catheter combinations are typically utilized as dilatation devices for dilating a body lumen, e.g., a coronary artery, or other body cavity, and have also been used in other capacities, such as for fixation and occlusion, e.g., for temporarily anchoring an instrument within a body lumen so that a surgical or therapeutic procedure can be performed. Other patents generally showing the application of various types of balloon catheter combinations include U.S. Pat. No. 4,540,404 issued to Wolvek, U.S. Pat. No. 4,422,447 issued to Schiff, and U.S. Pat. No. 4,681,092 issued to Cho et al. Exemplary applications for balloon and catheter combinations include angioplasties, carpal tunnel dilation, billiary dilation, urethral dilation, benign prostate hyperplasia (BPH) treatment, Barrett's esophagus treatment, fallopian tube dilation, tear duct dilation, valvuloplasty, etc.

Inflation and deflation of the balloon(s) 1604 is controlled by the control circuitry 906. When inflated, the balloon(s) 1604 create drag, and/or apply pressure or generate friction with respect to the adjacent wall of the alimentary tract where the capsule 1600 is located. Applications and instances in which it would be advantageous to apply brakes for slowing or stopping traversal of the capsule 1600 include procedures for taking an image with a camera on board the capsule, for administering a payload of medicament carried on board the capsule, for sensing ambient conditions, for taking a sample of ambient fluid, delivering phototherapeutic drugs, performing light therapy in conjunction with the phototherapeutic drugs, and for performing a diagnostic or therapeutic procedure.

The balloon(s) 1604 are selectively inflatable and deflatable. In FIG. 16, balloon 1604A is shown in an inflated state, and balloon 1604B is shown in a deflated state. FIG. 17 shows region 1700 in greater detail, in which a pressurizing closure member 1606 is shown, which is provided between the gas pressurization module 1602 and an associated balloon 1604 for selectively allowing a one-directional flow of gas from the gas pressurization module 1602 to the balloon 1604. Depressurizing closure member 1608 is further provided for selectively allowing a one-directional flow of gas from an associated balloon 1604 through an associated exhaust channel 1610 for allowing deflation of the balloon 1604 by allowing gas to exit the balloon 1604 through the exhaust channel 1610 and into the ambient environment of the capsule 1600.

Operationally, the balloon(s) 1604 may be inflated or deflated at a selected time or location, or in accordance with a sensed property or instructions from a remote processing device or another capsule. Inflation of the balloon may be used to stop, slow or steer the capsule's progress through the alimentary tract. The capsule 1600 may include additional one or more devices for performing a therapeutic or diagnostic procedure. After the treatment, the balloon(s) 1604 may be fully or partially deflated for allowing the capsule 1600 to continue traversing the alimentary tract, after which the balloon(s) may selectively re-inflated, such as for repeating the procedure at a different location along the alimentary tract.

The respective balloon(s) 1604 may be mounted on the capsule 1600. FIG. 17 shows an exemplary flange 1612 formed on housing 102 to which balloon 1604 is secured for mounting. The elasticity of the balloon 1604 causes the balloon 1604 to squeeze the neck of balloon 1614 with a force against the flange 1612 for maintaining the balloon 1604 secured. Additional structural features for securing the neck 1614 to the flange 1612 may be provided with the neck 1614 or flange 1612, such as ridges, ribs, mating grooves or notches, etc.

The respective balloon(s) 1604 may be secured to the capsule 1600 in a variety of ways. For example, a respective balloon 1604 may include an elastic strap or pouch attached to the balloon 1604 or integral therewith which grasps the housing 102 in addition to or instead of flange 1612. The tension due to elasticity of the strap/pouch holds the balloon 1604 in position. The housing, the neck of the balloon 1614 or the strap/pouch could be provided with additional securing mechanisms, such as ribs, mating grooves or notches, etc. The strap/pouch may be configured to accommodate other features of the capsule 1600, such as having apertures, e.g., for dispensing of medicament and/or for accommodating the antenna 502. Methods and structures known in the art, such as a balloon catheter combination may further be mounted to capsule 1600, e.g., the catheter is mounted to the capsule and the balloon 1604 is mounted to the catheter. The catheter may extend only slightly from the housing 102.

FIG. 19 shows capsule 1900 with a balloon and catheter combination, where operation of the capsule 1900 is similar to operation of capsule 1600. The capsule 1900 is a free standing capsule which is not attached structurally to a device located external to the patient. A balloon 1901 and catheter 1904 are provided inside a temporary housing 1903, which is controllably discarded from the capsule 1900 after ingestion of the capsule 1900. The discarded housing 1903 is dissolved, absorbed and/or passed through the alimentary tract for exit thereof. The control circuitry 906 and gas pressurization module 1602 are disposed within the catheter 1904 or a lumen of the balloon (e.g., where the balloon has multiple lumens). The gas pressurization module 1602 is in fluid communication with the balloon 1901 via channels 1906 and pressurization closure members 1606. Depressurization closure member 1608 is in fluid communication with the balloon 1901 and an exhaust channel 1610 through the catheter 1901 for allowing gas to exit the balloon 1901 through the exhaust channel 1610. The positioning of the closure members 1606 and 1608 may be changed for positioning the closure members 1606 and 1608 elsewhere and is not limited to the example shown.

The housing 1903 is made of a biocompatible material, such as a material that melts away or dissolves after ingestion due to a biochemical process in the alimentary tract. Preferably, the melting process is controlled, as known in the art, for discarding the housing 1903 at a desired location. It is further contemplated that the housing 1903 melt away from the catheter balloon combination in response to one or more events controlled by the control circuitry. The event may include the heating of one or more electrodes for melting the housing 1903, or release of a chemical stored internally to the housing 1903, where the chemical triggers the melting process. Once the housing 1903 is removed, the catheter balloon combination is exposed to the alimentary tract. The catheter 1904 and/or the balloon 1901 are rounded at their ends for passing safely through the alimentary tract without causing damage thereto.

With respect to FIGS. 16-19, the control circuitry controls the gas pressurization module 1602, the pressurization closure member 1606 and the depressurization closure member 1608 for controllably and repeatably inflating and deflating the balloons 1901 or 1604, such as in accordance with an event, such as a timed event, a sensed event (e.g., sensed pressure exceeding or falling below a predetermined threshold value) and/or a received command from an external device, such as a remote processing device or another capsule. The external device, for example, may track the capsule 1600 and/or monitor sensed conditions and/or timing events, and send control signals to the capsule 1600 for controlling inflation and deflation of the balloon(s) 1604 or 1901.

The description of the balloon(s) 1604 herein applies to balloon 1901. The balloon(s) 1604 may be of the high-pressure, non-elastic variety, which are formed of materials such as flexible polyvinyl chloride (PVC), cross linked polyethylene (PE), polyester polyethylene terephthalate (PDT), Nylon, or polyurethane; or the low-pressure elastomeric variety, which are formed of materials such as latex or silicone. Coatings on the balloon may be provided, such as selected from at least one of lubricious coatings (e.g., hydropholic, hydrophobic), abrasion and puncture resistant coatings, tacky or high friction coatings, conductive coatings, anti-thrombogenic coatings, drug release coatings, reflective coatings and selective coatings.

It is envisioned that the capsule 1600 may include one or more controlled vacuum or negative pressurized chambers for deflating of the balloon(s) 1604 and holding gas that exits from the deflated balloon(s) 1604. A compressor may be supplied with the capsule 1600 for compressing air within the vacuum chamber for reducing the size thereof. In the preferred embodiment, the vacuum chamber is not provided. Deflation is facilitated by opening one or more closure members, such as the depressurizing closure member 1608 for allowing gas in a respective balloon 1604 to exit controllably through the associated exhaust channel 1610. When the depressurizing closure member 1608 is opened, gas in the associated balloon 1604 will exit through the exhaust channel 1610 due to the tendency for pressure to normalize relative to ambient conditions and/or due to pressure exerted by the patient's anatomy, such as by muscles along the alimentary tract, e.g., due to peristaltic action.

The deflated balloon 1604B, such as before inflation and/or after deflation, may crumple into a random shape or collapse into a shape defined by structural features provided with the material of the balloon, such as predetermined creases, ribs and/or the equivalent. The deflated balloon 1604B may be packed and/or secured to the housing 102 or inside the capsule 1600 when not in use.

The pressurizing closure member 1606 and depressurizing closure member 1608 selectably allow a fluid, more specifically a gas, to flow in only one direction. Preferably, the rate of flow is controllable by adjusting an opening of the closure member 1608 and/or pressure at which the fluid is provided to the closure member 1606 or 1608. Selective opening, closing and preferably degree thereof, of closure members 1606 and 1608 is preferably provided by control circuitry 906. Closure members 1606 and 1608 may be similar functionally and structurally to closure member 966 described above, and may include a MEMS valve, a microvalve and microvalve actuator mechanism, a fluistor, a microfluidic system, a hatch, a micromotor and/or a controllable artificial muscle.

With respect to FIGS. 16-18, pressurizing closure member 1606 is in fluid communication with aperture 1802 in housing 102 which provides a passage between the gas pressurizing module 1602 and the associated balloon 1604. The depressurizing closure member 1608 is in fluid communication with aperture 1804 in housing 102 which provides a passage between the associated balloon 1602 and the exhaust channel 1610. The housing is further provided with an aperture 1806 for providing access from the exhaust channel 1610 to the ambient surroundings of the capsule 1600. With respect to FIG. 19, pressurizing closure member 1606 is in fluid communication with an aperture in balloon 1901 which provides a passage between the gas pressurizing module 1602 and the balloon 1901. The depressurizing closure member 1608 is in fluid communication with an aperture in balloon 1901 which provides a passage between the balloon 1901 and the exhaust channel 1610 which opens to the ambient surroundings of the capsule 1900.

The gas pressurizing module 1602 stores at least one starter element and generates gas therefrom, preferably pressurized gas, for inflating balloon(s) 1604 or 1901. The balloon(s) 1604 or 1901 may be provided with one or more regulators and/or pressure sensors 1620 for regulating and sensing the amount of pressure in the balloon(s) 1604 1901 or outside of the balloon(s) 1604 or 1901. Output from the pressure sensor(s) 1620 may be included in signals processed by the control circuitry 906 for determining when to release pressurized gas into or out of the balloon(s) 1604 or 1901. In one embodiment of the disclosure, the gas pressurizing module 1602 may include a canister for storing compressed gas, which may be similar to an air horn or scuba tank. Small canisters for holding CO2 and having small nozzles are known for remote controlled model airplanes. The gas may include, for example, nitrogen, CO2, helium, neon, argon, krypton, xenon, and/or radon. A preferred gas is argon, due do its pH neutrality, non-toxicity, lack of radiation and noninterference with electrical functions, so as not to interfere with biological functions when released through the exhaust channel 1610 into the alimentary tract or with electrical functions of the capsule 1900, however the disclosure is not limited thereto.

The gas is fed through the pressurizing closure member 1606 for inflating the balloon(s) 1604 or 1901 by controlling the closure member 1606 and/or the gas pressurizing module 1602 (e.g., an actuator thereof) by the control circuitry 906. The gas may be provided to the balloon(s) 1604 or 1901 intermittently. Accordingly, the balloon(s) 1604 or 1901 may be inflated and deflated multiple times.

The diameter of the alimentary tract and the shape of the inflated balloon(s) 1604 or 1901 are factors used to determine the desired volume of gas once delivered to the balloon. Exemplary diameters for an alimentary tract are as follows:

Small Intestines: 2.5 cm diameter

Large Intestines: 6.3 cm diameter

Esophagus: 2.5 cm diameter

Changes in volume of gas under pressure may be understood, for example, using the Boyle's Law and/or the ideal gas law, which is derived from Boyle's law and Charles' law.

Boyle's Law states:

P₁V₁=P₂V₂,

where the variables with the 1 subscript mean initial values before a manipulation (e.g., of pressure) and the variables with the 2 subscript mean final values after the manipulation.

Using Boyle's Law, an exemplary calculation is performed for a pressurization of 830 psi within the canister, and assuming rafts are about 2 psi and the atmosphere is about 15 psi:

830/(2+15)=48.8

Accordingly, such pressurization would provide an expansion of about 50 times the original volume of a compressed gas. Adjustments would be made for factors, such as temperature, atmospheric pressure, safety measures, initial volume of liquid gas, desired volume of generated gas, and cooling of generated gas with the initial rapid expansion (which typically quickly reach ambient temperature).

From the above, it is evident that a tiny amount of liquid gas, such as nitrogen or CO2, may be stored in a small ingestible canister, where pressurization thereof will generate a gas for inflating one or more balloons to a size appropriate for slowing, stopping or steering the capsule 1600 or 1900 within the portion of the alimentary tract it is traversing. The degree to which the balloon(s) 1604 are inflated depends on factors such as the patient's anatomy, the patient's age, the patient's body temperature, atmospheric pressure and the balloon configuration being used. Inflation of the balloon(s) 1604, 1901 may also be controlled based on results from an imaging system and/or a tracking system which can determine that the capsule 1600 or 1900 has stopped, confirming that sufficient pressure has been reached in the balloon (s) 1604, 1901 to stop the capsule 1600, 1900. Accordingly, parameters of the treatment, such as the degree of pressurization, actuation of the pressurization, and control of the closure member(s) 1606 are controlled according to the above factors. Information related to the above factors may be provided to the control circuitry 906 or the remote processing device 950 before beginning the procedure (e.g., as pretreatment data) and/or during the treatment (e.g., after the capsule 1600 has been ingested). The remote processing device may consult a knowledge base or database for determining additional information based on information already provided. For example, a knowledge base or database may provide information relating to alimentary tract diameters for a patient of a particular age, weight and height.

The gas pressurizing module 1602 may alternatively include an electrolytic cell, such as described by U.S. Pat. No. 5,318,557 issued to Gross, in which an electric current is applied to the electrolytic cell for generating a gas. Alternatively, the gas pressurizing module 1602 may include two or more chemicals in a solid, gas or liquid state, which react when combined to produce a gas. An example of such a gas pressurizing module is embodied in a car airbag, wherein a very small amount of powder or solid propellant (e.g., sodium azide and potassium nitrate) reacts to produce nitrogen very quickly upon an electrical trigger.

In the capsule 1600 or 1900 the gas is preferably discharged to the balloon(s) 1604 or 1901 gently without great speed and/or force. It is preferable to use non-toxic chemicals. However, the chemicals used to generate the gas are contained within the capsule and expelled with the capsule from the patient, preferably without exposing the patient's anatomy to the chemicals. Accordingly, it is contemplated that toxic chemicals could be used. Actuation of the trigger to cause generation of the gas is controlled by the control circuitry 906, as described above with respect to actuation of the canister.

It is further contemplated that the capsules 1600 or 1900 may include more than one gas pressurizing module 1602 for inflating the balloon(s) 1604 or 1901. For example, when one of the gas pressurizing modules 1602 is depleted, another one will take over for inflating the balloon(s). Alternatively, a first and second gas pressurizing module may each be in fluid communication with a different balloon 1604.

Special features of balloons and catheter balloons which are known in the art may be applied to the balloon(s) 1604 and/or to the catheter balloon configuration of FIG. 19 which includes catheter 1902 and balloon 1902. Furthermore, as described above, balloon 1604 may be embodied as a catheter balloon, where the catheter is mounted to the capsule 1600.

As described, for example in U.S. Pat. No. 5,342,301 issued to Saab, a perimetrical lumen 1630 may be provided, wound around the outer wall of the balloon(s) 1604 or 1901, such as in a helical pattern. The perimetrical lumen 1630 may include pinholes 1631 along its length, and may be used to precisely deliver medicament at a selected time or location of the capsule 1600. The capsule 1600 may include a medicament dispensing system, such as system 901, and the perimetrical lumen 1630 is connected to an output of the medicament dispensing system 901. Dispensing of the medicament through the lumen 1630 is controlled, such as by controlling closure members and/or a pressure mechanism of the medicament dispensing system 901. With respect to FIG. 19, the medicament system 901 may further be disposed within a lumen of balloon 1901 (not shown) and is in fluid communication with the perimetrical lumen 1630 wound around balloon 1901.

The balloons 1604 or 1901 may be provided with multiple lumens, such as for performing multiple functions. The multiple lumens may hold different devices, such as diagnostic or therapeutic devices, and may further be used for precise positioning.

The capsules 1600 or 1900 may further be provided with a microwave antenna. The microwave antenna may be disposed inside the balloons 1604 or 1901 for application of microwave energy through the walls of the balloon for heating tissue, or may be disposed inside housing 102A, where at least a portion of the housing 102 is formed of a material that is appropriate for transferring heat from the microwave antenna to the outside surface of the housing 102. A cooling system, such as a cooling balloon may be provided for cooling the antenna and/or tissue not targeted for heating.

The capsules 1600 or 1900 may further be provided with a laser or infrared delivery device 1640 mounted therein, such as for laser balloon dilation and photo dynamic therapy (PDT) with light activated (phototherapeutic) drugs, such as Photofrin™, ALA, 5-ALA, Foscan™, Metex, e.g., for the treatment of Barrett's esophagus or infrared activated drugs. The inflated PDT balloon expands the esophagus and positions the laser or infrared delivery device 1640. The laser or infrared delivery device 1640 may be disposed inside the balloons 1604 or 1901 for application of light energy through the walls of the balloon to the tissue, or the laser or infrared delivery device 1640 may be disposed inside housing 102. The balloon(s) 1604 or 1901 or a portion of housing 102 (e.g., a window 1642 shown in phantom) are translucent for allowing passage of light or infrared energy from the laser or infrared delivery device 1640 to the environment of the tissue. Furthermore, the balloon(s) 1604 or 1901 or a portion of housing 102 may be provided with an opaque coating at selected positions for preventing light from passing there through or an infrared resistant coating for preventing infrared energy from passing through for preventing treating tissue that is not targeted for light therapy. Furthermore, the laser or infrared delivery device 1640 may be provided embedded in or external to housing 102.

The capsule 1600 or 1900 may include two discrete balloons 1604 or 1901 disposed at opposite ends of the capsule (or catheter 1902) or a dog bone shaped balloon, a medicament delivery system and/or a suction system. When the opposing balloons 1604 or 1901 are both inflated, an area between the two balloons 1604 or 1901 is sealed off from the rest of the alimentary tract. The sealed off area may be treated, such as by administering a medicament, e.g., a toxic medicament. After treatment, the suction system may suck excess medicament from the area. A second medicament may be administered for flushing out the area. The balloons 1604 or 1901 are then deflated for allowing the capsule 1600 or 1901 to pass through and exit the alimentary tract.

The balloons 1604 or 1901 may be provided with a microporous membrane with holes ranging in sizes ranging from submicron to a few microns in diameter. The membrane can be infused or impregnated with a medicament, wherein upon stretching of the membrane, such as upon inflation of the balloon 1604 or 1901, the medicament is more easily released. The balloon membrane seeps medicament for dispensing the medicament in very precise doses over a well-defined area. Furthermore, medicament may be coated onto the surface of the balloons 1604, 1901 and delivered to a specific site. Pressure, heat, laser light, etc., may facilitate transfer of the medicament from the balloon's surface to the wall of the alimentary tract.

A first capsule and second capsule may operate in tandem. The first capsule includes balloons 1604 or 1901, and may be used to block passage of the second capsule for positioning of the second capsule or to block flow of a medicament past the first capsule. The second capsule may or may not include balloons or a balloon catheter. The second capsule may perform a diagnostic or therapeutic treatment. Upon completion of the treatment, the balloons of the first capsule are deflated and both capsules may continue traveling the alimentary tract. The procedure is repeatable for multiple discreet and intermittent treatments.

FIG. 20 shows a capsule 2000 having a plurality of bristles 2002 attached to the capsule 2000 and distributed about the capsule 2000, preferably 360 degrees around the circumference of cross-sections of the capsule, preferably near the back end of the capsule which trails the front end during traversal of the alimentary track. The capsule 2000 is a free standing capsule which is not attached structurally to a device located external to the patient. The bristles are formed of a biocompatible material that is biased to extend away from the capsule, such as at an angle that is less than 90 degrees. The length of the bristles is sufficient so that as the capsule traverses the alimentary tract the bristles contact the wall of the alimentary tract. As the wall changes shape, the deflections of the individual bristles change. A deflection sensor 2004 is provided for the respective bristles for sensing the degree of the deflection and sending a corresponding signal to a control circuitry 906. The control circuitry 906 stores the signals corresponding to the deflection and/or transmits the signals to the remote processing device 950, such as via antenna 502. The signals corresponding to the deflection are processed for generating a topical mapping of the alimentary tract, such as for identifying anomalies.

The front end and the back end of the capsule 2000 are tapered, with the back end preferably tapered more severely. As the capsule 2000 traverses the alimentary tract the capsule 2000 expands the alimentary tract in places where it may be collapsed. The bristles extend from the capsule adjacent to or at the cross-section where its diameter is greatest and extend backwards at an angle towards the tapered end. The bristles brush along the alimentary tract before it has returned to a collapsed state, but having sufficient room to be deflected due to the severely tapered backend.

The deflection sensors 2004 may be placed interior or exterior to the housing 102 of the capsule 2000, or may be disposed in an aperture in the housing 102. Preferably, each sensor is positioned on an exterior face of the housing 102 at the location where a corresponding bristle is attached to the housing 102 or exits the housing 102. The bristles may extend through the housing 102 at corresponding apertures, where the apertures are sealed so that no fluid passes there through.

The deflection sensors 2004 communicate with the control circuitry 906, such as by wired or wireless communication. Where the sensors 2004 are placed exterior to the housing 102, wired connections for communication between the respective deflection sensors 2004 and the control circuitry 906 pass through at least one aperture, where the aperture is sealed so that no fluid passes there through. Furthermore, any portion of wired connections situated exterior to the housing are impervious to fluid.

In addition to or instead of the bristles 2002 and the deflection sensors 2004, the capsule 2000 may be provided with pressure sensors 2010 which sense pressure exerted against them and generate corresponding sensed pressure signals which are received by the control circuitry 906, such as by wired or wireless communication. The control circuitry 906 stores or transmits the pressure signals. The pressure signals are processed for generating a pressure mapping of the alimentary tract, such as for identifying anomalies.

The density of the bristles 2002 and pressure sensors 2010 is selected in accordance with design choice. The plurality of bristles 2002 or pressure sensors 2010 may include a single row or several rows of strategically places bristles 2002 or pressure sensors 2010, respectively. The processing of the deflection signals may include sampling and/or detecting and processing changes in deflection. Advantageously, the capsule 2000 can examine topographic features and pressure exertion features of the entire alimentary tract without an invasive procedure. Even areas of the alimentary tract that are difficult to access by endoscopy or colonoscopy are mapped by the capsule 2000.

With respect to FIG. 21, a capsule 2100 for administering radiation controllably is shown. Disposed within capsule 2100 is a radioactive material, such as Iodine-125 or Palladium-103. The capsule 2100 may be ingestible for traversal of the alimentary tract, where traversal of the capsule 2100 is controlled, e.g., stopped or slowed, for positioning the capsule at a target region for administering the radiation to a targeted region without radiating a region that is not targeted. Traversal of the alimentary tract by the capsule 2100 is controlled preferably by a brakes mechanism on the capsule 2100, such as the balloons 1604 or 1901 as shown and described with respect to FIGS. 16-19. Furthermore, traversal of the alimentary tract by the capsule 2100 may be controlled by administering a medicament (e.g., which is dispensed via the capsule or another dispensing means) for slowing or stopping peristaltic action, such as Lomotil®, in addition to or instead of using the brakes mechanism. Alternatively, the capsule 2100 may be implantable, such as for implantation at a desired location adjacent a target, such as a tumor. The capsule 2100 is a free standing capsule which is not attached structurally to a device located external to the patient.

The capsule 2100 includes an adjustable shield, wherein when the position of the shield is adjusted to a closed position the environment of the capsule 2100 is shielded from radiation. Furthermore, adjustment of the position of the shield is controllable to an open position for providing a gap or opening that provides fluid communication between the radioactive material and environment of the capsule for allowing the environment of the capsule to be exposed to radiation. The size of the opening is selectable for controlling the amount of radiation released from the capsule 2100. Additionally, the capsule, including the shield is configurable for providing the openings in a desirable arrangement for directing the radiation in one or more selected directions.

Advantages of the capsule 2100 include minimization of radiation exposure to non-target entities, such as to a medical team handling the capsule prior to ingestion, to non-targeted tissue, or targeted tissue when radiation exposure is not desired; the ability to release the radiation intermittently, and/or over a long period of time, such as in accordance with a remote or embedded control program which may provide for adjustment of the treatment depending upon the response of the tumor or lesion and/or the condition of the patient; the ability to administer radiation to selected locations along the alimentary tract from within the alimentary tract for minimizing exposure of non-targeted tissue to radiation.

With reference to FIGS. 21-29, exemplary capsule 2100 and its congruent variation 2100′ are shown. FIG. 21 shows an exploded view of the radiation capsule 2100, where a main body 2102 and an adjustable module 2104 of the capsule 2100 are shown. In the example shown, the module 2104 is rotated for adjusting its position. It is contemplated that other structures and methods may be used for adjusting the position of the module 2104, such as sliding, telescoping, expanding, contracting, etc., and the present disclosure is not limited to rotation of the module 2104.

The main body 2102 includes a first half 2102A for housing components of the capsule 2100, such as control circuitry and an actuator as described below, and a second half 2102B for housing a radioactive assembly 2106 which includes a radioactive material 2107, as described further below. The first half 2102A of the main body 2102 includes a housing 2108 enclosing the first half 2102A, and a radiation resistant control housing 2110 for enclosing components of the capsule 2100, such as the control circuitry and actuator and protecting the same from emitted radiation. As shown in the exemplary configuration of FIG. 21, the housing 2108 and the control housing 2110 may be one entity, where the control housing 2110 houses the first half 2102A, including components of the capsule 2100, such as the control circuitry and actuator.

The second half of main body 2102B includes at least one first radiation resistant panel 2116, where multiple first panels 2116 may converge and are preferably attached to first end cap 2118 having an aperture 2120. Gaps 2122 are formed in between adjacent first panels 2116. The radioactive assembly 2106 preferably includes a solid material 2154, such as a biocompatible plastic shell, which is mounted to the inside face of the first panels 2116 and is preferably exposed at the gaps 2112. Mounted in the solid material 2154 are radioactive grains or seeds (which include the radioactive material 2107). Preferably the seeds are strategically placed on the solid material 2154 for being positioned in the gaps 2122. Alternatively, as shown in FIG. 24, the radioactive material 2107 may be mounted on a solid material 2154 which is supported within the second half 2102B by a first support assembly 2112, and is exposed through gaps 2122 to the ambient environment of the capsule 2100. The housing 2108, the resistant first panels 2116 and/or the first end cap 2118 may be formed of an integral piece of material, or may be formed of separate pieces of material that are coupled together, such as snapped together. Accordingly, the first and second halves of the main body 2102A and 2102B may be formed of one piece of material or multiple pieces of material. It is preferable that in the embodiment in which the resistant first panels 2116 and the housing 2108 are formed of one piece of material, the housing 2108 includes the control housing 2110.

The second half of the main body 2102B is not limited to the configuration of first panels 2116 shown. Other limitations of the main body 2102B may be provided in which a first radiation resistant assembly is provided having at least one radiation resistant portion, e.g., a panel, with at least one gap formed within the first assembly. For example, the first assembly may include one panel having a gap described therein. Alternatively, multiple panels may be provided in which at least one gap is described between the panels or within the respective panels.

FIG. 22 shows a cross-sectional view of first half of the main body 2102A in which the control housing 2110 is supported within the housing 2108 by a second support assembly 2124. The hatched area shown is the inside wall 2128 of the housing 2108. A rotational device 2126, such as a shaft, is operationally attached at a first end of the rotational device 2126 to the actuator disposed within control housing 2110. Upon activation or enablement of the actuator, the rotational device 2126 is rotated. The rotational device 2126 is received, supported and rotatable at a second end of the rotational device 2126 within the aperture 2120 of the first end cap 2118 of the second half of the main body 2102B.

The module 2104 includes at least one second radiation resistant panel 2136, where multiple second panels 2136 may converge and are preferably attached to second radiation resistant end cap 2138 which receives and supports rotational device 2126. The second end cap 2138 further functions to prevent any radiation which passed through aperture 2120 from exiting the capsule 2100. The second panels 2136 and the second end cap 2138 may be formed of one integral piece of material or may be formed of multiple pieces of material. The second end cap 2138 may include an interior second coupling mechanism (not shown) for receiving the rotational device 2126 without allowing rotation within the coupling mechanism. For example, the rotational device 2126 may be welded to, snapped into, or screwed into the second end cap 2138. Gaps 2142 are formed in between adjacent second panels 2136. When the capsule 2100 is assembled, the module 2104 fits over the main body 2102. Upon activation, the actuator turns the rotational device 2126 for causing the module 2104 to rotate, which causes the module 2104 to rotate about the main body 2102. Preferably the surfaces of at least one of the main body 2102 and the module 2104 which face one another when assembled together are coated with a material, such as Teflon™, which minimizes friction as the module 2104 moves with respect to the main body 2102/

The module 2104 is not limited to the configuration of second panels 2136 shown. Other limitations of the module 2104 may be provided in which a second radiation resistant assembly is provided having at least one radiation resistant portion, e.g., a panel, and at least one gap 2142. The position of a respective second panel 2136 of the at least one second pane 21361 is adjustable to a position with respect to a respective gap 2122 of the at least one gap 2122 for selectively covering at least a portion of the respective gap 2122 for impeding passage of radiation through the respective gap 2122 to the ambient environment of the capsule 2100. It is contemplated that one second panel 2136 may cover one or more gaps 2122.

Similarly, the position of a respective gap 2142 of the at least one gap 2142 is adjustable to a position with respect to a respective gap 2122 for selectively exposing the gap 2122 to the ambient environment of the capsule 2100. The at least one second panel 2136 is operatively coupled to the actuator 2160 as shown in FIG. 23, for adjusting the position of the at least one second panel 2136 and the at least one gap 2142, such as via rotation, sliding, telescoping, expanding, contracting, etc., and the present disclosure is not limited to rotation of the module 2104.

Assembly of the capsule 2100 is performed by fitting the module 2104 over the main body 2102 and inserting the rotational device 2126 into the end cap 2138 so that the module 2104 is supported by the rotational device 2126. Alternatively, the rotational device 2126 may be fixedly attached to the end cap 2138 of the module 2104 and inserted through the control housing 2110 and into the actuator 2160, e.g., motor, where it is received for supporting and rotating the rotational device 2126. The rotational device 2126 may be removable at either of its ends, and assembly may include placing one of its ends in the assembled position and then the other end, where the order of which of the ends is placed first is in accordance with design choice.

It is contemplated that the module 2104, when assembled, be positioned inside the main body. Whether the module 2104 fits over or inside the main body 2102, activation of the actuator 2160 causes rotation of the module 2104 while the main body 2102 does not rotate, e.g., remains stationary. Preferably, friction associated with rotation of the module 2104 is minimized, such as by providing a gap between the main body 2102 and the second panels 2136 of the module 2104.

Accordingly, it is preferable that a cross-sectional slice from top 2140 to bottom 2141 of the main body 2102 or the module 2104 be a circle, and that when assembled, at any point along the length of the capsule 2100, the diameter of the cross-section of the module 2104 is greater than the cross-section of the main body 2102. Furthermore, it is preferable, particularly for the embodiment shown in FIG. 21, that the length and the width of the second panels 2136 of the module 2104 exceed the length and the width of the first panels 2116 of the main body 2102, respectively, so that when assembled the second panels 2136 overlap the first panels 2116 in the width and length thereof for providing maximum radiation resistance for preventing radiation from exiting the capsule 2100 when the capsule is in a closed position, as described further below.

The control housing 2110, the second panels 2136 and the first panels 2116 each include a layer of radiation resistant material, such as lead, which impedes passage of radiation through the control housing 2110 or first or second panels 2116, 2316. The outer surface of the capsule 2100, which includes the outer surface of the second panels 2136, first panels 2116, the control housing 2110, and/or the housing 2108 includes a coating that is biocompatible, such as the materials used for the housing 102, e.g., derivatives of polyether urethane and/or other biocompatible polymers for preventing leakage of lead into the body of the patient.

The second support assembly 2124 supports the control housing 2110 within the capsule 2100, where preferably the control housing 2110 is suspended at a central location of the capsule so that the actuator 2160, e.g., motor, is strategically positioned for receiving the rotational device 2126. For the configuration of FIG. 21 in which the control housing 2110 is included with the housing 2108, the actuator 2160 is supported by the second support assembly 2124 for strategically positioning the actuator 2160, as described above.

The control circuitry and/or other components of the capsule 2100 (e.g., a power supply, communication circuitry, etc.) may be further supported by the second support assembly 2124 or another support assembly. The components of the capsule 2100 other than the radioactive assembly 2106, e.g., the actuator, control circuitry, communication circuitry, etc., may be disposed in one or more housings which may be nested or distinct and separated, provided that those components which could potentially be negatively affected by radiation are protected from the radiation by radiation resistant housings. The rotational device 2126 exits the control housing 2110 through a gap therein. Accordingly, adequate radiation resistant protection material is provided at the gap for preventing penetration of radiation even with the rotational device 2126 inserted there through for not allowing radiation to penetrate the control housing 2110 through the gap.

Power for one or more components of the capsule 2100 may be supplied actively, such as by a power supply on board the capsule 2100, such as a lithium battery. It is contemplated that the capsule 2100 does not include a power supply and that power is supplied to one or more components of the capsule 2100 by a device that couples energy to the capsule 2100 for providing energy thereto.

An exemplary second support assembly 2124 is shown in FIG. 23. The second support assembly 2124 is secured at first and second ends 2126 and 2148, respectively, to the housing 2108 or to one or more first panels 2116. The second support assembly 2124 includes a C clamp 2150 for holding the control housing 2110. The C clamp 2150 may be further attached to the housing 2108 or a first panel 2116 for providing further mechanical stability.

The radioactive material 2107 of the radioactive assembly 2106 is preferably suspended within a solid material 2154, such as a plastic that does not degrade with exposure to radiation. The radioactive assembly 2106 is preferably positioned near the gaps 2122 and not immediately behind the first panels 2116. For example, the radioactive assembly 2106 may be located along a longitudinal axis of the capsule 2100. It is advantageous to minimize the distance traversed by radiation emitted from the radioactive assembly 2106 for minimizing attenuation of the radiation before it reaches its target. Accordingly, it is contemplated that the radioactive assembly 2106 may include two or more assemblies strategically positioned and supported at different locations, where the locations are preferably offset from the longitudinal axis of the capsule 2100 in order to be proximate the gaps 2122.

The first support assembly 2112 includes at least one support structure for supporting the radioactive assembly 2106 in the desired at least one position as described above. The support structures may be attached to opposing first panels 2116 and include at least one C clamp for holding the radiation assembly 2112 in the desired position.

FIG. 23 shows actuator 2160, communication circuitry 504, ultrasound transducer element 510a and control circuitry 906 which may be disposed within the control housing 2110 for protection from radiation emitted by the radioactive assembly 2106. Other components of capsule 2100 may further be disposed within control housing 2110, and the actuator 2160 and the control circuitry 906 could be disposed within separate radiation resistant housings. The actuator 2160 includes one or more devices, such as a micromotor, which are capable of facilitating adjustment of the position of the at least one second panel 2136, e.g., by rotating rotational device 2126. For example, the actuator 2160 may be a piezoelectric motor, also known as an ultrasonic motor, which is known to be reliable, small and have low power consumption. Other types of actuators may be used, such as actuators that operate in response to a thermal, light, electrical, acoustical, chemical, etc., stimulation, and which facilitate adjustment of the at least one second panel 2136, such as by causing an element to rotate, slide, expand, contract, etc.

Communication circuitry 504 and/or ultrasound transducer element 510a may be provided for facilitating communication between the capsule 2100 and another device remote from the capsule, such as a remote processing device external to the patient or another capsule having a communication capability (such as any of the capsules described in this disclosure or as are known in the art).

The control circuitry 906 provides control signals to the actuator 2160 for controlling activation of the actuator 2160. As described above, the control circuitry 906 includes timing circuitry and mechanisms and/or circuitry for starting and/or controlling the timing circuitry, as well as any interfaces for interfacing with other components of the capsule 2100, such as the actuator 2160 or communication circuitry. The control circuitry 906 controls the actuator in response to signals received from a remote device (e.g., a remote processing device or another capsule) via antenna 502 and/or communication circuitry; sensor information from sensors (e.g., as shown in the embodiment of FIG. 9A); and/or timing information. It is contemplated that more than one actuator 2160 may be provided for working in tandem with each other to rotate the rotational device 2126. It is preferable that at least a portion of the control circuitry is disposed within the capsule 2100, but is not limited thereto. It is contemplated, as described above with respect to FIG. 9A, that at least a portion of the control circuitry 906 is located external to the patient and sends control signals which are received by the actuator, such as via antenna 502.

FIGS. 24-26 show a capsule 2100′, which is congruent with capsule 2100, but differs from capsule 2100 in that first panels 2116 and the second panels 2136 of module 2104 extend virtually along the entire length of the capsule 2100′. FIG. 24 shows a cross-sectional side view of the main body 2102 of capsule 2100′, in which it is shown that the control housing 2110 is provided internal to the capsule 2100′ and its housing 2108. A first end cap 2118 is provided for supporting the rotational device 2126 and one end of the rotational device 2126. The inner face of first panels 2116 is shown as hatched.

FIG. 25 shows a perspective view, with the rotation device 2126 shown in phantom, of another embodiment of the main body 2102 of the capsule 2100′ in which first end caps 2118 are provided at opposing ends of the capsule 2100′, and the rotational device 2126 extends between the two first end caps 2118. The rotational device 2126 (shown in phantom) exits the control housing 2110 at two locations, both of which are adequately shielded for not allowing radiation to penetrate the control housing 2110. Support of the rotational device by the two first end caps 2118 provides additional mechanical stability. The inside face of first panel 2116 is shown as hatched. FIG. 26 shows the module 2104 of the capsule 2100′, which includes opposing second end caps 2138 for securing to opposite ends of the rotational device 2126, respectively, and for providing shielding to radiation for not allowing radiation to exit from inside the capsule 2100′ through the second end caps 2138. In operation, the assembled capsule 2100′ emits radiation when in an open position omnidirectionally.

FIG. 27 shows en end view of assembled capsule 2100 in a fully opened position and FIG. 28 shows an end view of assembled capsule 2100 in a fully closed position. The control circuitry controls activation of the actuator 2160 for opening or closing the capsule 2100, or partially opening the capsule so that it assumes a position somewhere between the positions shown in FIGS. 27 and 28.

Accordingly, in operation, when the capsule 2100 is in a closed position the environment of the capsule 2100 is shielded from radiation emitted by the radiation assembly 2106 by virtue of the overlapping second panels 2136 of the module 2104. Once implanted or ingested the control circuitry may actuate the actuator 2160 for causing the capsule to assume an open position, a closed position or a position therebetween in response to an event, such as a timed event, a sensed event or instructions from a remote device, such as a remote processing device external to the patient or another capsule, such as capsule of one of the embodiments described herein or as known in the art.

The described embodiments of the present disclosure are intended to be illustrative rather than restrictive, and are not intended to represent every embodiment of the present disclosure. Various modifications and variations can be made without departing from the spirit or scope of the disclosure as set forth in the following claims both literally and in equivalents recognized in law.

1. An ingestible electrical capsule system (1400) for acquiring samples along the alimentary tract of a patient comprising:
a housing (102) having at least one aperture (970);
at least one impervious collection chamber (1402) disposed within said housing (102) and having at least one aperture in fluid communication with a respective aperture of the at least one aperture (970) of the housing (102);
at least one closure member (966), individual closure members of the at least one closure member (966) associated with a respective aperture of the at least one collection chamber (1402), wherein the individual closure members are actuatable between an open state for permitting flow of the fluid through the respective closure member into the associated collection chamber (1402) for acquiring a sample of ambient fluid, and a closed state for substantially blocking flow of fluid into and out of the associated collection chamber (1402) for storing the acquired sample; and
control circuitry (906) for controlling actuation of said at least one closure member (966).

2. The capsule system (1400) according to claim 1, wherein the individual closure members are further actuatable from a closed state to an open state for acquiring a sample at a selected time.

3. The capsule system (1400) according to claim 1, wherein the control circuitry (906) controls the at least one closure member (966) in accordance with at least one predetermined condition selected from the group of conditions consisting of a sensed condition, a timed condition and receipt of an instruction from a remote processor.

4. The capsule system (1400) according to claim 1, wherein prior to opening a closure member associated with a collection chamber (1402), a negative pressure is provided interior to the collection chamber (1402).

5. The capsule system (1400) according to claim 1, wherein the capsule system (1400) further comprises a weighted element (1430) disposed within the housing (102) or attached to the housing (102) for orienting the capsule (1400) during transversal of the alimentary tract.

6. The capsule system (1400) according to claim 1, wherein respective closure members (966) comprise an artificial muscle including a polymer which selectably contracts and expands in response to an electrical stimulus for achieving the open and closed states of the respective closure member (966).

7. The capsule system (1400) according to claim 1, wherein respective closure members of said at least one closure member (966) are addressable for independent control of actuation thereof.

8. The capsule system (1400) according to claim 1, wherein the control circuitry (906) further controls the size of an opening of respective closure members (966).

9. The capsule system (1400) according to claim 1, further comprising a sensor (904) disposed within respective collection chambers (1402) of the at least one collection chamber (1402) for sensing the presence of fluid within the collection chamber (1402) for triggering actuation of the at least one closure member (966) associated with the collection chamber (1402) for closing the at least one closure member (966).

10. The capsule system (1400) according to claim 1, wherein respective apertures of the at least one aperture (970) of the housing (102) are oriented so that during traversal of the alimentary tract by the capsule system (1400) the current of flow of fluid within the alimentary tract assists entry of the fluid into a collection chamber of the at least one collection chamber (1402) in fluid communication with the respective apertures.

11. The capsule system (1400) according to claim 1, further comprising:
at least one expulsion sensor (904) for sensing expulsion or imminent expulsion of the capsule system (1400) from the alimentary tract and generating corresponding signals;
an alert device (1440) for selectively generating an alert;
wherein the control circuitry (906) processes the corresponding signals generated by the expulsion sensor (904) for determining when the corresponding signals are indicative of sensed expulsion or imminent expulsion, and controls the alert device (1440) to generate the alert when it is determined that the corresponding signals indicate sensed expulsion or imminent expulsion.

12. The capsule system (1400) according to claim 1, wherein the alert device (1440) is disposed within the housing (102).

13. The capsule system (1400) according to claim 1, wherein the expulsion sensor (904) is selected from the group of sensors consisting of pressure, light and temperature sensors for sensing a change in sensed light, temperature or pressure.

14. The capsule system (1400) according to claim 1, wherein the alert device (1440) further comprises at least one of an audio device for generating a sound alert, a light source for generating a glowing light alert, and a medicament dispensing system for dispensing an alert medicament having a distinguishable characteristic selected from the group of characteristics consisting of color and odor.

15. The capsule system (1400) according to claim 1, wherein the alert device (1440) comprises a vibrational device for generating a vibration alert.

16. The capsule system (1400) according to claim 1, wherein the system (1400) acquires a series of samples and the housing (102) is provided with a marking for indicating which closure member (1406) of the at least one closure member (966) provides access to the first sample acquired of the series of samples acquired.

17. An ingestible electrical capsule system (1400) for traversing the alimentary tract of a patient comprising:
a housing (102);
at least one expulsion sensor (904) for sensing expulsion or imminent expulsion of the capsule system (1400) from the alimentary tract and generating corresponding signals;
an alert device (1440) for selectively generating an alert; and
control circuitry (906) for processing the corresponding signals generated by the expulsion sensor (904) for determining when the corresponding signals are indicative of sensed expulsion or imminent expulsion, and controlling the alert device (1440) to generate the alert when it is determined that the corresponding signals indicate sensed expulsion or imminent expulsion.

18. The capsule system (1400) according to claim 17, wherein the alert device (1440) is disposed within the housing (102).

19. The capsule system (1400) according to claim 17, wherein the expulsion sensor (904) is selected from the group of sensors consisting of pressure, light and temperature sensors for sensing a change in sensed light, temperature or pressure.

20. The capsule system (1400) according to claim 17, wherein the alert device (1440) further comprises at least one of an audio device for generating a sound alert, a light source for generating a glowing light alert, and a medicament dispensing system for dispensing an alert medicament having a distinguishable characteristic selected from the group of characteristics consisting of color and odor.

21. The capsule system (1400) according to claim 17, wherein the alert device (1440) comprises a vibrational device for generating a vibration alert.

22. A method for generating an alert traversing upon expulsion or imminent expulsion from the alimentary tract of a device traversing the digestive tract comprising the steps of:
sensing expulsion or imminent expulsion of the device from the alimentary tract and generating corresponding signals;
processing the corresponding signals;
determining when the corresponding signals are indicative of sensed expulsion or imminent expulsion, and
generating an alert when it is determined that the corresponding signals indicate sensed expulsion or imminent expulsion.

23. The method according to claim 22, wherein the generating the alert step is performed from within a housing of the device.

24. The method according to claim 22, wherein the sensing step includes sensing a change in sensed light, temperature or pressure.

25. The method according to claim 22, wherein the generating the alert step comprises at least one of generating a sound alert, generating a glowing light alert, and dispensing an alert medicament having a distinguishable characteristic selected from the group of characteristics consisting of color and odor.

26. The method according to claim 22, wherein the generating the alert step comprises generating a vibration alert.
